(19) United States
(12) Patent Application Publication
(10) Pub. No.: US 2007/0172839 A1
(43) Pub. Date: Jul. 26, 2007

(54) Asymmetrical adapters and methods of use thereof

(76) Inventors: Douglas R. Smith, Gloucester, MA (US); Joel A. Malek, Beverly, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/338,620

(22) Filed: Jan. 24, 2006

(51) Int. Cl. C12Q 1/68; C12P 19/34; C07H 21/04

(52) U.S. Cl. 435/6; 435/91.2; 536/24.3

(57) ABSTRACT

A pair of asymmetrical, partially double-stranded oligonucleotide adapters are provided wherein the pair of adapters comprise a first asymmetrical oligonucleotide adapter comprising a single-stranded 3′ overhang and a second asymmetrical double-stranded oligonucleotide adapter comprising a single-stranded 5′ overhang and at least one blocking group on the strand of said second asymmetrical oligonucleotide adapter that does not comprise the 5′ overhang. Also provided are a pair of double-stranded Y oligonucleotide adapters and a pair of double-stranded bubble oligonucleotide adapters and methods of using said asymmetrical adapters for amplification of at least one double stranded nucleic acid molecule, wherein the amplification produces a plurality of amplified nucleic acid molecules having a different nucleic acid sequence at each end are also described. Also provided is a method for exponentially amplifying one strand in a double-stranded nucleic acid molecule. Also provided are methods for preparing libraries of paired tags using COS-linkers. Also provided are cleavable adapters comprising an affinity tag and a cleavable linkage, wherein cleaving the cleavable linkage produces two complementary ends. Methods of using the cleavable adapters to produce a paired tag library are also described.

### GOVERNMENT SUPPORT

The invention was supported, in whole or in part, by a grant HG003570 from the National Institutes of Health. The Government has certain rights in the invention.

### BACKGROUND OF THE INVENTION

Sequencing of nucleic acid molecules derived from complex mixtures (e.g., mRNA populations) or entire genomes (e.g., a prokaryotic or eukaryotic genome) by a shotgun approach requires specific strategies for fragmenting and manipulating the starting nucleic acid molecules in order to facilitate accurate reconstruction of the sequences of those molecules. In the traditional whole genome sequencing strategy, the starting DNA is fragmented into smaller pieces in a variety of different size ranges (e.g., insert sizes of 2 kb, 10 kb, 40 kb and 150 kb) and cloned into vectors allowing replication and amplification in a bacterial host (e.g., high copy number plasmid, low copy number plasmid, fosmid and BAC vectors for propagation of the different insert sizes in E. coli). Although this approach has been successfully applied to many genomes, it invariably results in numerous gaps in the final reconstructed sequence after assembly at typical redundancy levels (e.g., 6-10× sequence coverage). This is caused by non-random sequence representation in the starting libraries resulting from loss of certain sequences during the shotgun cloning procedure, a phenomenon known as cloning bias. Clone based, or hybrid approaches to whole genome sequencing utilizing collections of pre-mapped bacterial artificial chromosome (BAC) clones has been advocated as an alternative to the whole genome shotgun method, but is no longer considered a cost-effective alternative.

Classical DNA sequencing techniques, such as the Maxam and Gilbert chemical cleavage method (Maxam and Gilbert, 1977, Proc. Natl. Acad. Sci. USA 74: 560-564; incorporated herein by reference) and the Sanger chain termination method (Sanger et al. 1977, Proc. Natl. Acad. Sci. USA 74: 5463-5467; incorporated herein by reference) are cumbersome and inefficient. Several alternative sequencing approaches that utilize massively parallel amplification or surfaces or on individual microbeads from millions of molecules in a single reaction vessel have been described in recent years. Although it is possible to produce short fragments suitable for PCR amplification and paired end sequence generation, efficient methods for doing so from long DNA fragments have not been described.

Thus, a pressing need exists for alternatives to conventional cloning procedures, which can be used, for example, to generate paired-end sequences from genomic or mRNA derived fragments.

### SUMMARY OF THE INVENTION

The present invention provides asymmetrical oligonucleotide adapters which can be used for the exponential amplification of a nucleic acid sequence wherein the resulting amplified product will have a different nucleic acid sequence on each end. In addition, the asymmetrical adapters permit the exponential amplification of a single strand from a double-stranded nucleic acid sequence. The present invention also provides methods for the generation of paired end libraries of DNA fragments wherein the paired ends are derived from the ends of DNA molecules about 2-200 kb in size.

Sequencing nucleic acid molecules derived from complex mixtures (e.g., mRNA populations) or entire genomes (e.g., a prokaryotic or eukaryotic genome) by a shotgun approach requires specific strategies for fragmenting and manipulating the starting nucleic acid molecules in order to facilitate accurate reconstruction of the sequences of those molecules. However, the current methods have a number of disadvantages. For example, the traditional whole genome sequencing strategy suffers from cloning bias which results in numerous gaps in the final reconstructed sequence, clone-based, or hybrid approaches using collections of pre-mapped bacterial artificial chromosome (BAC) clones is not cost-effective, classical DNA sequencing techniques, such as the Maxam and Gilbert chemical cleavage method (Maxam and Gilbert, 1977, Proc. Natl. Acad. Sci. USA 74: 560-564; incorporated herein by reference) and the Sanger chain termination method (Sanger et al. 1977, Proc. Natl. Acad. Sci. USA 74: 5463-5467; incorporated herein by reference) are cumbersome and inefficient, and alternative sequencing approaches that use massively parallel amplification reactions on surfaces or on individual microbeads from millions of molecules in a single reaction vessel all rely on PCR-based template generation procedures as currently practiced. Efficient methods for producing short fragments suitable for PCR amplification and paired end sequence generation from long DNA fragments have not been described.

Because of these limitations, there is a pressing need for alternatives to conventional cloning procedures which can be used, for example, to generate paired-end sequences from genomic or mRNA derived fragments. Such alternatives are provided herein and enable the construction of truly random fragment libraries in a wide range of size classes (e.g., about 2 kb, 5 kb, 10 kb, 50 kb, 100 kb or 200 kb with a narrow window of size variation within each class) in a suitable format for DNA sequencing and without any prior passage through a bacterial host. The randomness of fragment end points is important to complete genome assembly without gaps. Libraries produced by means of fragmentation with restriction endonucleases, which have been disclosed previously (e.g., in U.S. Pat. No. 6,054,276, U.S. Pat. No. 6,720,179 and WO03/074734), are not sufficiently random because the occurrence of restriction endonuclease cleavage sites is sparse, sequence dependent, highly variable and non-random in nature. Methods described herein also provide a reliable means to amplify genomic DNA fragments with high fidelity, e.g., by polymerase chain reaction (PCR), in such a way as to ensure that each amplified fragment ends up with a different (unique) universal primer sequence at each end. This is desirable in some of the methods described herein because a variety of the sequencing technologies that utilize massively parallel amplification reactions on beads or surfaces from millions of molecules in a single experiment utilize a template generation strategy that requires a different universal priming site at each end of the starting DNA fragments. In addition, methods described herein allow amplification of a single strand from a double-stranded nucleic acid sequence to facilitate, e.g., heterozygosity analysis or characterization of hemi-methylation status.

Thus, the present invention provides compositions and methods to achieve those ends, as well as providing methods useful for whole genome single nucleotide polymorphism (SNP) discovery, genotyping, karyotyping, and characterization of insertions, deletions, inversions, translocations and copy number polymorphisms.

The present invention provides asymmetrical oligonucleotide adapters (also referred to herein as asymmetrical adapters, asymmetrical linkers, cap adapters, unistrand adapters or unistrand linkers), which can be used to amplify a nucleic acid molecule (e.g., a double stranded nucleic acid molecule), wherein the amplification produces a plurality of amplified nucleic acid molecules having a different nucleic acid sequence at each end. In a particular embodiment, the present invention is directed to a pair of asymmetrical oligonucleotide adapters. In another particular embodiment, the pair of asymmetrical oligonucleotide adapters are not identical such that in an amplification reaction, one strand of a double-stranded nucleic acid sequence having a first and second non-identical asymmetrical adapter at either end (also referred to herein as an end-linked nucleic acid molecule or sequence) is selectively and/or exponentially amplified. For example, an amplification reaction of an end-linked nucleic acid molecule, wherein the end-linked nucleic acid molecule comprises a first asymmetrical adapter at one end, and a second, non-identical, asymmetrical adapter at the other end, the amplification reaction comprises amplifying one strand of the end-linked nucleic acid molecule referred to herein as the template strand. The amplification reaction comprises (1) a first primer that is complementary to a primer binding site in a first asymmetrical adapter in the template strand. The first primer is contacted with the template strand under conditions in which a first nucleic acid strand is synthesized in the amplification reaction, wherein the first nucleic acid strand is complementary to the full length of the template strand, and wherein the 3′ end of the first nucleic acid strand comprises a second primer binding site that is complementary to a sequence in the second asymmetrical adapter in the template strand. The amplification reaction further comprises (2) contacting the first nucleic acid strand with a second primer that is complementary to the second primer binding site in the first nucleic acid strand under conditions in which a complementary strand of the first nucleic strand is synthesized. In one embodiment, the steps of contacting the first primer and the second primer can be done simultaneously. In another embodiment, the steps of contacting the first primer and the second primer can be done sequentially. As will be understood by a person of skill in the art, these amplification steps are repeated to exponentially amplify a template strand. As used herein, a “first primer” or a “second primer” refers to a plurality of first primer molecules or a plurality of second primer molecules. In one embodiment, the plurality of first primer molecules comprise identical nucleic acid sequences and/or the plurality of second primer molecules comprise identical nucleic acid sequences. In another embodiment the plurality of first primer molecules comprise different nucleic acid sequences and/or the plurality of second primer molecules comprise different nucleic acid sequences. In a particular embodiment, the plurality of first primers bind to the same first primer binding site and/or the plurality of second primers bind to the same second primer binding site.

As used herein, two (or more) asymmetrical adapters are “non-identical” or “not identical” when the asymmetrical adapters differ from each other by at least one nucleotide in a primer binding site, by at least one nucleotide in the complementary nucleic acid sequence of a primer binding, and/or by the presence or absence of a blocking group. Furthermore, the two (or more) non-identical asymmetrical adapters can have substantial differences in nucleic acid sequences. For example, two asymmetrical tail adapters, asymmetrical bubble adapters or two asymmetrical Y adapters (described in more detail below) can comprise entirely different sequences (e.g., with little or no sequence identity). In a particular embodiment, the non-identical asymmetrical adapters have little or no sequence identity in the unpaired region (e.g., the tail region, the arms of the Y region, or the bubble region). Alternatively, a pair of asymmetrical adapters are not identical such that they differ in kind or type, e.g., the first and second asymmetrical adapters are not both asymmetrical tail adapters, not both asymmetrical Y adapters, or not both asymmetrical bubble adapters. That is, a pair of asymmetrical adapters can comprise, e.g., an asymmetrical tail adapter and a bubble adapter or Y adapter, or a pair of asymmetrical adapters can comprise a bubble and a Y adapter. In a particular embodiment, two (or more) asymmetrical adapters that are not identical in kind or type differ from each other by at least one nucleotide in a primer binding site, by at least one nucleotide in the complementary nucleic acid sequence of a primer binding, and/or by the presence or absence of a blocking group.

In one embodiment a pair of asymmetrical adapters comprises a pair of tail oligonucleotide adapters (also referred to herein as tail adapters, 3′ tail adapter and 5′ tail adapter, asymmetrical tail adapters, asymmetrical oligonucleotide adapters, asymmetrical adapters, “JamAdapters”, “JamLinkers” and variations thereof). A pair of tail adapters comprises: (a) a first oligonucleotide adapter which comprises a 3′ overhang (or tail); and (b) a second oligonucleotide adapter which comprises a 5′ overhang (or tail) with at least one blocking group at the 3′ end of the strand that does not comprise the 5′ tail. In a particular embodiment, the first and second tail adapters are not identical. In another particular embodiment, at least one end of the tail adapter is a ligatable end. In another particular embodiment, the 3′ overhang of the first asymmetrical tail adapter comprises at least one primer binding site. In a further particular embodiment, the 3′ overhang of the first asymmetrical tail adapter and the 5′ overhang of the second asymmetrical tail adapter are each at least about 8 nucleotides to at least about 100 nucleotides in length. In yet another particular embodiment, the 3′ overhang of the first asymmetrical tail adapter and the 5′ overhang of the second asymmetrical tail adapter are each at least about 25 nucleotides to at least about 40 nucleotides in length. In another particular embodiment, a tail adapter of the present invention is at least about 15 nucleotides to at least about 100 nucleotides in length. In another particular embodiment, a tail adapter of the present invention is at least about 50 nucleotides to at least about 75 nucleotides in length.

In another embodiment, provided herein is a pair of asymmetrical adapters, wherein each asymmetrical adapter in the pair comprises a Y oligonucleotide adapter (also referred to herein as Y adapter, asymmetrical Y adapter, asymmetrical adapter or asymmetrical oligonucleotide adapter). A pair of asymmetrical Y oligonucleotide adapters comprise: (a) a first (partially double-stranded) Y oligonucleotide adapter comprising a first ligatable end, and a second unpaired end which comprises two non-complementary strands, wherein the two non-complementary stands cause the unpaired end to form the arms of a “Y” shape; and (b) a second (partially double-stranded) Y oligonucleotide adapter comprising a first ligatable end, and a second unpaired end which comprises two non-complementary strands, wherein the two non-complementary stands cause the unpaired end to form the arms of a “Y” shape. In a particular embodiment, the first and second asymmetrical Y oligonucleotide adapters are not identical. The length of the non-complementary strands in each Y adapter can be the same or different. In one embodiment, the length of the non-complementary strands in either or both of the first or second Y oligonucleotide adapter are at least about 8 nucleotides in length. In another embodiment, the non-complementary strands are at least about 8 nucleotides to at least about 100 nucleotides in length. In another embodiment, the non-complementary strands are at least about 25 nucleotides to at least about 40 nucleotides in length. In one embodiment, an asymmetrical Y adapter of the present invention is at least about 15 nucleotides to at least about 100 nucleotides in length. In another embodiment, an asymmetrical Y adapter of the present invention is at least about 50 nucleotides to at least about 75 nucleotides in length. In one embodiment, at least one non-complementary strand of the first (and/or second) Y adapter comprises at least one primer binding site.

In another embodiment, a pair of asymmetrical adapters comprises a pair of bubble oligonucleotide adapters (also referred to herein as bubble adapters, asymmetrical bubble adapters, asymmetrical adapters or asymmetrical oligonucleotide adapters). A pair of asymmetrical bubble oligonucleotide adapters comprise: (a) a first (partially double-stranded) bubble oligonucleotide adapter comprising at least one unpaired region flanked on each side by a paired region; and (b) a second (partially double-stranded) bubble oligonucleotide adapter comprising at least one unpaired region flanked on each side by a paired region, wherein the first and second asymmetrical bubble oligonucleotide adapters are not identical. In one embodiment, the length of the unpaired region in each bubble adapter is the same or different. In another embodiment, the length of the unpaired region in each strand of a bubble adapter is the same or different. In a particular embodiment, the length of the unpaired region in either or both bubble adapters is at least about 8 nucleotides in length. In another particular embodiment, the unpaired regions is at least about 5 nucleotides to at least about 25 nucleotides in length. In a further embodiment, the length of the unpaired regions is at least about 8 nucleotides to at least about 15 nucleotides in length. In a further embodiment, one or more bubble adapters comprises more than one unpaired region. In one embodiment, an unpaired region in the first (and/or second) bubble adapter comprises at least one primer binding site.

Also provided herein is a method for amplification of at least one double-stranded nucleic acid molecule. In a particular embodiment, amplification produces a plurality of amplified molecules having a different sequence at each end. In another embodiment, exponential amplification is of one strand of a double-stranded nucleic acid molecule. As illustrated in FIGS. 1A-1C, 2A-2C, 3A-3C and 4A-4C, the method comprises ligating to one end of the double-stranded nucleic acid molecule a first asymmetrical adapter selected from the group consisting of:
(i) an asymmetrical tail adapter comprising a first ligatable end, and a second end comprising a single-stranded 3′ overhang of at least about 8 nucleotides;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region.

The method further comprises ligating to the other end of the double-stranded nucleic acid molecule a second asymmetrical adapter selected from the group consisting of:
(i) an asymmetrical tail adapter comprising a first ligatable end, and a second end comprising a single-stranded 5′ overhang of at least about 8 nucleotides, wherein the 3′ end of the strand that does not comprise the 5′ overhang comprises at least one blocking group;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region.

In the method, the first and second asymmetrical adapters are not identical which provides for the exponential amplification of one strand of the double-stranded nucleic acid molecule in an amplification reaction. Non-identical first and second asymmetrical adapters also provide for the amplification of nucleic acid molecules having a different sequence at each end.

When an asymmetrical adapter is ligated to each end of the double-stranded nucleic acid molecule, an end-linked double-stranded nucleic acid molecule is produced. The method further comprises amplifying one strand of the end-linked nucleic acid molecule referred to herein as the template strand. The amplification reaction comprises (1) contacting the template strand with a first primer that is complementary to a first primer binding site in a first asymmetrical adapter in the template strand. Under appropriate conditions, the first primer synthesizes a first nucleic acid strand in the amplification reaction, wherein the first nucleic acid strand is complementary to the template strand, and wherein the 3′ end of the first nucleic acid strand comprises a second primer binding site that is complementary to a sequence in the second asymmetrical adapter in the template strand. The amplification reaction further comprises (2) contacting the first nucleic acid strand with a second primer that is complementary to the second primer binding site in the first nucleic acid strand under conditions in which a complementary strand of the first nucleic acid strand is synthesized. The amplification steps (1) and (2) are repeated, and the amplification produces a plurality of amplified molecules having a different sequence at each end (see, e.g., FIGS. 2A-2C, 3A-3C and 4A-4C for a schematic illustration).

In another aspect of the invention, a pair of asymmetrical oligonucleotide adapters comprises a pair of asymmetrical adapters wherein the first and second asymmetrical adapter are not identical in kind (e.g., as discussed above, the first and second asymmetrical adapters are not both asymmetrical tail adapters, or both asymmetrical Y adapters, or both asymmetrical bubble adapters) and are selected from the group consisting of:
(i) an asymmetrical tail adapter comprising a first ligatable end, and a second end comprising a single-stranded 3′ overhang of at least about 8 nucleotides;
(ii) an asymmetrical tail adapter comprising a first ligatable end, and a second end comprising a single-stranded 5′ overhang of at least about 8 nucleotides, wherein the 3′ end of the strand that does not comprise the 5′ overhang comprises at least one blocking group;
(iii) an asymmetrical Y adapter comprising a first ligatable end, and a second unpaired end comprising two non-complementary strands, wherein the length of the non-complementary strands are at least about 8 nucleotides; and
(iv) an asymmetrical bubble adapter comprising an unpaired region of at least about 8 nucleotides flanked on each side by a paired region.

The pair of asymmetrical adapters can be used in a variety of methods, such as amplification of at least one double stranded nucleic acid molecule. In a particular embodiment, amplification produces a plurality of amplified nucleic acid molecules having a different nucleic acid sequence at each end. When the asymmetrical adapters are ligated to each end of the double-stranded nucleic acid molecule, an end-linked double-stranded nucleic acid molecule is produced. Thus, the method further comprises amplifying one strand of the end-linked nucleic acid molecule referred to herein as the template strand. The amplification reaction comprises (1) contacting the template strand with a first primer that is complementary to a first primer binding site in a first asymmetrical adapter in the template strand. Under appropriate conditions, the first primer synthesizes a first nucleic acid strand in the amplification reaction, wherein the first nucleic acid strand is complementary to the template strand, and wherein the 3′ end of the first nucleic acid strand comprises a second primer binding site that is complementary to a sequence in the second asymmetrical adapter in the template strand. The amplification reaction further comprises (2) contacting the first nucleic acid strand with a second primer that is complementary to the second primer binding site in the first nucleic acid strand under conditions in which a complementary strand of the first nucleic acid strand is synthesized. The amplification steps (1) and (2) are repeated, and the amplification produces a plurality of amplified molecules having a different sequence at each end.

In a further aspect of the invention, provided herein is a method for producing and amplifying a paired tag from a first nucleic acid sequence fragment, without cloning. In the method, the 5′ and 3′ ends of a first nucleic acid sequence fragment are joined via a first linker such that the first linker is located between the 5′ end and the 3′ end of the first nucleic acid sequence fragment under conditions in which a circular nucleic acid molecule is produced (see, e.g., FIGS. 6 and 9). The circular nucleic acid molecule is cleaved, thereby producing a second nucleic acid sequence fragment (a paired tag) in which the 5′ end tag of the first nucleic acid sequence fragment is joined to the 3′ end tag of the first nucleic acid sequence fragment via the first linker (see, e.g., FIGS. 6 and 9). A pair of asymmetrical adapters are ligated to each end of the second nucleic acid sequence fragment (see, e.g., FIGS. 6 and 9). The pair of asymmetrical adapters comprise: a first asymmetrical oligonucleotide adapter selected from the group consisting of:
(i) an asymmetrical tail adapter comprising a first ligatable end, and a second end comprising a single-stranded 3′ overhang of at least about 8 nucleotides;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region,
   and a second asymmetrical oligonucleotide adapter selected from the group consisting of:
(i) an asymmetrical tail adapter comprising a first ligatable end, and a second end comprising a single-stranded 5′ overhang of at least about 8 nucleotides, wherein the 3′ end of the strand that does not comprise the 5′ overhang comprises at least one blocking group;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region.
   In the method, the first and second asymmetrical oligonucleotide adapters are not identical. When the second nucleic acid sequence fragment is ligated to the pair of asymmetrical adapters, an end-linked double-stranded nucleic acid sequence fragment is produced (see, e.g., FIGS. 1A-1C). The method further comprises amplifying one strand of the end-linked nucleic acid molecule referred to herein as the template strand. The amplification reaction comprises (1) contacting the template strand with a first primer that is complementary to a first primer binding site in a first asymmetrical adapter in the template strand. Under appropriate conditions, the first primer synthesizes a first nucleic acid strand in the amplification reaction, wherein the first nucleic acid strand is complementary to the template strand, and wherein the 3′ end of the first nucleic acid strand comprises a second primer binding site that is complementary to a sequence in the second asymmetrical adapter in the template strand. The amplification reaction further comprises (2) contacting the first nucleic acid strand with a second primer that is complementary to the second primer binding site in the first nucleic acid strand under conditions in which a complementary strand of the first nucleic acid strand is synthesized. The amplification steps (1) and (2) are repeated, and amplifies the end-linked nucleic acid molecule (the paired tag), thereby producing and amplifying a paired tag from a first nucleic acid sequence fragment without cloning (see, e.g., FIGS. 2A-2C, 3A-3C and 4A-4C).

In one embodiment of the method, the first linker employed to join the 5′ and 3′ ends of a first nucleic acid sequence fragment as described herein comprises at least one affinity linker. An affinity linker, as used herein, comprises two ligatable ends and affinity tag. Examples of an affinity tag include biotin, digoxigenin, a hapten, a ligand, a peptide and a nucleic acid. The affinity linker thus introduced provides a means to purify the circularized molecules in which the 5′ and 3′ ends of the first nucleic acid sequence fragment have been joined together, and to purify nucleic acid sequence fragments that have been cleaved to produce paired tags prior to amplification.

In a still further aspect of the invention provided herein is a method for characterizing a nucleic acid sequence, without cloning. The method comprises fragmenting a nucleic acid sequence thereby producing a plurality of first nucleic acid sequence fragments, each having a 5′ end and a 3′ end. The 5′ and 3′ ends of each first nucleic acid sequence fragment are joined to a first linker such that the first linker is located between the 5′ end and the 3′ end of each first nucleic acid sequence fragment in a circular nucleic acid molecule (see, e.g., FIGS. 6 and 9). The plurality of circular nucleic acid molecules are cleaved, thereby producing a plurality of second nucleic acid sequence fragments wherein at least a portion of the fragments comprise a paired tag derived from each first nucleic acid sequence fragment joined via the first linker. A pair of asymmetrical adapters are ligated to both ends of each second nucleic acid sequence fragments, wherein the pair of asymmetrical adapters comprise: a first asymmetrical oligonucleotide adapter selected from the group consisting of:
(i) an asymmetrical tail adapter comprising a first ligatable end, and a second end comprising a single-stranded 3′ overhang of at least about 8 nucleotides;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region,
   and a second asymmetrical oligonucleotide adapter selected from the group consisting of:
(i) an asymmetrical tail adapter comprising a first ligatable end, and a second end comprising a single-stranded 5′ overhang of at least about 8 nucleotides, wherein the 3′ end of the strand that does not comprise the 5′ overhang comprises at least one blocking group;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region.
   In the method, the first and second asymmetrical oligonucleotide adapters are not identical. When the pair of asymmetrical adapters are ligated to each end of each second nucleic acid sequence fragments a plurality of end-linked nucleic acid sequence fragments is produced. The method further comprises amplifying one strand of the end-linked nucleic acid molecule referred to herein as the template strand. The amplification reaction comprises (1) contacting the template strand with a first primer that is complementary to a first primer binding site in a first asymmetrical adapter in the template strand. Under appropriate conditions, the first primer synthesizes a first nucleic acid strand in the amplification reaction, wherein the first nucleic acid strand is complementary to the template strand, and wherein the 3′ end of the first nucleic acid strand comprises a second primer binding site that is complementary to a sequence in the second asymmetrical adapter in the template strand. The amplification reaction further comprises (2) contacting the first nucleic acid strand with a second primer that is complementary to the second primer binding site in the first nucleic acid strand under conditions in which a complementary strand of the first nucleic acid strand is synthesized. The amplification steps (1) and (2) are repeated, and the amplification reaction amplifies the end-linked nucleic acid molecules (the second nucleic acid fragments), thereby producing a plurality of amplified second nucleic acid fragments containing a different sequence at each end. The method further comprises characterizing the 5′ and 3′ end tags of the plurality of amplified second nucleic acid fragments.

In another aspect of the invention provided herein is a method for producing a paired end library (also referred to herein as a paired tag library) from a nucleic acid sequence. In one embodiment, the nucleic acid sequence is a genomic DNA sequence. In one embodiment, the paired ends derive from nucleic acid sequence fragments approximately 48 kb +/−about 5 kb in size. The method comprises fragmenting a nucleic acid sequence to produce a plurality of nucleic acid sequence fragments of an appropriate size which can be packaged into lambda bacteriophage heads. As will be understood by a person of skill in the art, the appropriate size of a nucleic acid fragment for packaging into a lambda bacteriophage head is approximately 48 kb +/−about 5 kb in size. A plurality of linkers, each comprising a functional lambda bacteriophage packaging (COS) site, are ligated to the plurality of nucleic acid sequence fragments under conditions in which concatemers of the nucleic acid sequence fragments with intervening COS site linkers are produced (see, e.g., FIG. 11). Individual nucleic acid sequence fragments containing a bacteriophage COS linker at each end in the same orientation in the concatemers are maintained under conditions in which they are packaged into bacteriophage particles (see FIG. 11). A plurality of packaged, circularized COS-linked nucleic acid sequences, wherein the ends of each nucleic acid sequence fragment are linked by a nicked COS site, are produced. As will be understood by a person of skill in the art, a nicked COS site is the result of the packaging wherein two COS sites in the same orientation are cleaved to produce complementary ends which anneal (hybridize) to each other (but still contain a nicked sugar-phosphate backbone in the nucleic acid sequence at the junctions of the annealed complementary ends) to form a circularized COS-linked nucleic acid sequence, and wherein each circularized COS-linked nucleic acid sequence is packaged into a single bacteriophage particle. The circularized COS-linked nucleic acid sequences are liberated from the bacteriophage particles under conditions wherein the nicked COS sites remain annealed (and thus, the COS-linked nucleic acid sequence remains circularized). The nicked COS site in each circularized COS-linked nucleic acid sequence are ligated with DNA ligase under conditions suitable for ligation of the nicked COS sites to produce a plurality of closed circular COS-linked nucleic acid sequences. The plurality of closed circular COS-linked nucleic acid sequences are fragmented under conditions in which at least a portion of the fragments contain the COS linker flanked on both sides with at least a portion of the nucleic acid sequence (a COS-linked paired end comprising a nucleic acid sequence “tag” from each end (5′ end and 3′ end) of the nucleic acid sequence and the COS linker linking the two tags: e.g., which can be schematically represented as: 5′ end tag-COS-3′ end tag), thereby producing a paired end library from a nucleic acid sequence comprising COS-linked paired ends.

In a preferred embodiment, the COS-linkers further comprise an affinity tag (e.g., an affinity tag is biotin, digoxigenin, a hapten, a ligand, a peptide and a nucleic acid). The affinity tag can be used to purify the COS-linked nucleic acid sequence fragments after the fragmentation of the closed circular COS-linked nucleic acid sequences to remove fragments that do not contain a COS-linked paired end.

In one embodiment, the plurality of closed circular COS-linked nucleic acid sequences are fragmented by shearing. In a further embodiment, the plurality of closed circular COS-linked nucleic acid sequences that are fragmented by shearing are subsequently treated to produce blunt ends (also referred to herein as “blunt-ended” or “healed”). In another embodiment, the COS linker further comprises a restriction endonuclease recognition site for a restriction endonuclease. In a particular embodiment, the restriction endonuclease recognition site is recognized by a restriction endonuclease that cleaves a nucleic acid sequence distally to the restriction endonuclease recognition site (see, e.g., FIG. 12). Cleavage of the nucleic acid sequence distally to the restriction endonuclease recognition site produces a nucleic acid sequence tag. In a particular embodiment, the restriction endonuclease that cleaves a nucleic acid sequence distally to the restriction endonuclease recognition site is a TypeIIS and/or Type III restriction endonuclease. Thus, in one embodiment, the plurality of closed circular COS-linked nucleic acid sequences are fragmented by cleavage with a TypeIIS and/or Type III restriction endonuclease, wherein a paired tag is produced.

In another embodiment, the method for producing a paired end library from a nucleic acid sequence further comprises isolating the COS-linked nucleic acid sequence fragments. The isolated COS-linked nucleic acid sequence fragments can also be amplified to produce a library of amplified COS-linked nucleic acid sequence fragments. In one embodiment, the amplification comprises ligating a pair of asymmetrical adapters to the ends of each COS-linked nucleic acid sequence fragment, wherein the pair of asymmetrical adapters comprise:

a first asymmetrical oligonucleotide adapter selected from the group consisting of:

(i) an asymmetrical tail adapter comprising a first ligatable end, and a second end comprising a single-stranded 3′ overhang of at least about 8 nucleotides;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region,
   and a second asymmetrical oligonucleotide adapter selected from the group consisting of:
(i) an asymmetrical tail adapter comprising a first ligatable end, and a second end comprising a single-stranded 5′ overhang of at least about 8 nucleotides, wherein the 3′ end of the strand that does not comprise the 5′ overhang comprises at least one blocking group;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region.
   In the method, the first and second asymmetrical oligonucleotide adapters are not identical. When a pair of asymmetrical adapters are ligated to each COS-linked nucleic acid sequence fragment, a plurality of end-linked nucleic acid sequence fragments is produced.

In one embodiment, the method further comprises amplifying one strand of the end-linked nucleic acid molecule referred to herein as the template strand. The amplification reaction comprises (1) contacting the template strand with a first primer that is complementary to a first primer binding site in a first asymmetrical adapter in the template strand. Under appropriate conditions, the first primer synthesizes a first nucleic acid strand in the amplification reaction, wherein the first nucleic acid strand is complementary to the template strand, and wherein the 3′ end of the first nucleic acid strand comprises a second primer binding site that is complementary to a sequence in the second asymmetrical adapter in the template strand. The amplification reaction further comprises (2) contacting the first nucleic acid strand with a second primer that is complementary to the second primer binding site in the first nucleic acid strand under conditions in which a complementary strand of the first nucleic acid strand is synthesized. The amplification steps (1) and (2) are repeated, and amplifies the end-linked nucleic acid fragments, thereby producing a plurality of amplified COS-linked nucleic acid fragments. In a further embodiment, the plurality of amplified COS-linked nucleic acid fragments are sequenced.

In another aspect of the invention, the method for producing a paired end library from a nucleic acid sequence comprises fragmenting a nucleic acid sequence to produce a plurality of nucleic acid sequence fragments of an appropriate size for packaging into a lambdoid bacteriophage head. A plurality of linkers, each comprising a functional lambda bacteriophage packaging (COS) site and two loxP sites flanking the functional COS site, are ligated to the plurality of nucleic acid sequence fragments under conditions in which concatemers of the nucleic acid sequence fragments with intervening COS site linkers are produced (see, e.g., FIG. 11). Individual COS-linked nucleic acid sequence fragments containing a bacteriophage COS linker at each end in direct repeat orientation in the concatemers are packaged into bacteriophage particles, under conditions in which a plurality of packaged, circularized COS-linked nucleic acid sequences, wherein the ends of each nucleic acid sequence fragment are linked by a nicked COS site are produced. The circularized COS-linked nucleic acid sequences are liberated from the bacteriophage particles under conditions that the nicked COS sites remain annealed. The nicked COS site in each circularized COS-linked nucleic acid sequence are sealed by ligation, (e.g., using DNA ligase such as T4 DNA ligase) to produce a plurality of closed circular COS-linked nucleic acid sequences. The plurality of closed circular COS-linked nucleic acid sequences are maintained under conditions suitable for intramolecular recombination between the two loxP sites in each closed circular COS-linked nucleic acid sequence, wherein intramolecular recombination between the two loxP sites removes the functional COS site from each closed circular COS-linked nucleic acid sequence fragments, and produces a plurality of closed, circular lox-linked nucleic acid sequences. The plurality of closed circular lox-linked nucleic acid sequences are fragmented (e.g., by shearing), thereby producing at least a portion of fragments comprising a nucleic acid sequence tag from each end of the nucleic acid sequence fragment linked by the recombined loxP site (i.e., lox-linked paired ends), thereby producing a paired end library from a nucleic acid sequence comprising lox-linked nucleic acid sequence fragments (see, e.g., FIG. 13). In one embodiment, the appropriate size for packaging of the nucleic acid fragments into a lambdoid bacteriophage head is at least about 48 kb +/−about 4 kb. In another embodiment, the COS-linkers further comprise an affinity tag. In a particular embodiment, the affinity tag is located outside of the loxP recombination sites in the COS linker (see, e.g., FIG. 13). An affinity tag can be selected from the group consisting of biotin, digoxigenin, a hapten, a ligand, a peptide and a nucleic acid. In one embodiment, the lox-linked nucleic acid sequence fragments are isolated by capturing the affinity tag. In another embodiment, the COS-linker further comprises a selectable marker. A selectable marker can be, for example, an antibiotic resistance gene or the like (e.g., a beta-lactamase to confer resistance to ampicillin, an aminoglycoside phosphotransferase to confer resistance to kanamycin or neomycin, a tetracycline efflux pump to confer resistance to tetracyclines, or a chloramphenicol acetyl transferase to confer resistance to chloramphenicol). In one embodiment, the selectable marker is located outside of the loxP recombination sites in the COS linker.

The plurality of closed circular lox-linked nucleic acid sequences can be fragmented in a variety of ways. In one embodiment, the plurality of closed circular lox-linked nucleic acid sequences are fragmented by shearing. In a particular embodiment, the fragments obtained from shearing the plurality of closed circular lox-linked nucleic acid sequences are subsequently blunt-ended. Blunt-ending of a nucleic acid sequence permits sequence-independent ligation to another nucleic acid sequence. In another embodiment, the COS linker further comprises a restriction endonuclease recognition site for a restriction endonuclease that cleaves a nucleic acid sequence distally to the restriction endonuclease recognition site. In one embodiment, the restriction endonuclease recognition site is located outside of the loxP recombination sites in the COS linker. Cleavage of a nucleic acid sequence distally to a restriction endonuclease recognition site produces a tag sequence. Cleavage of both ends of a nucleic acid sequence fragment distally to a restriction endonuclease recognition site produces paired tags (or paired ends) when linked together. The restriction endonuclease that cleaves a nucleic acid sequence distally to the restriction endonuclease recognition site can be a TypeIIS or Type III restriction endonuclease. Thus, in one embodiment, the plurality of closed circular lox-linked nucleic acid sequences are fragmented by cleavage with a TypeIIS or Type III restriction endonuclease. In a particular embodiment, the two loxP that flank the functional COS site in the COS-linker are mutated, whereby recombination between the two loxP sites is unidirectional (after recombination of the loxP sites, further recombination of the recombined lox site is inhibited or prevented). In one embodiment, the two loxP sites are a lox71 site and a lox66 site. In a further embodiment, the method for producing a paired end library from a nucleic acid sequence further comprises amplifying the isolated lox-linked nucleic acid sequence fragments, thereby producing a library of amplified lox-linked nucleic acid sequence fragments. Thus, in one embodiment, the amplification comprises ligating a pair of asymmetrical adapters to the ends of each lox-linked nucleic acid sequence fragment, wherein the pair of asymmetrical adapters comprise:

a first asymmetrical oligonucleotide adapter selected from the group consisting of:

(i) an asymmetrical tail adapter comprising a first ligatable end, and a second end comprising a single-stranded 3′ overhang of at least about 8 nucleotides;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region,
   and a second asymmetrical oligonucleotide adapter selected from the group consisting of:
(i) an asymmetrical tail adapter comprising a first ligatable end, and a second end comprising a single-stranded 5′ overhang of at least about 8 nucleotides, wherein the 3′ end of the strand that does not comprise the 5′ overhang comprises at least one blocking group;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region.
   In the method, the first and second asymmetrical oligonucleotide adapters are not identical. An end-linked nucleic acid sequence fragment is produced by ligating the pair of asymmetrical adapters to the lox-linked nucleic acid sequence fragment. The method further comprises amplifying one strand of the end-linked nucleic acid molecule referred to herein as the template strand. The amplification reaction comprises (1) contacting the template strand with a first primer that is complementary to a first primer binding site in a first asymmetrical adapter in the template strand. Under appropriate conditions, the first primer synthesizes a first nucleic acid strand in the amplification reaction, wherein the first nucleic acid strand is complementary to the template strand, and wherein the 3′ end of the first nucleic acid strand comprises a second primer binding site that is complementary to a sequence in the second asymmetrical adapter in the template strand. The amplification reaction further comprises (2) contacting the first nucleic acid strand with a second primer that is complementary to the second primer binding site in the first nucleic acid strand under conditions in which a complementary strand of the first nucleic acid strand is synthesized. The amplification steps (1) and (2) are repeated, and the amplification produces a plurality of amplified end-linked nucleic acid molecules (lox-linked nucleic acid fragments). In a further embodiment, the plurality of amplified lox-linked nucleic acid fragments are characterized. In a particular embodiment, the amplified lox-linked nucleic acid fragments are sequenced. In another embodiment, instead of a COS linker flanked by a pair of loxP sites, the COS linker is flanked by different site-specific recombination sites (e.g., a pair of frt sites, xer sites, or int sites).

In another aspect of the invention, provided herein is a cleavable adapter comprising an affinity tag and a cleavable linkage, wherein the cleavable linkage is not a restriction endonuclease cleavage site, and cleaving the cleavable linkage produces two complementary ends. In another embodiment, the affinity tag is selected from the group consisting of biotin, digoxigenin, a hapten, a ligand, a peptide and a nucleic acid. In a further embodiment, the cleavable adapter comprises a restriction endonuclease recognition site specific for a restriction endonuclease that cleaves a nucleic acid sequence distally to the restriction endonuclease recognition site. In another embodiment, the cleavable linkage in the cleavable adapter is a 3′ phosphorothiolate linkage. In another embodiment, the cleavable linkage in the cleavable adapter is a deoxyuridine nucleotide.

In another aspect of the invention, provided herein is a method for producing a paired tag library from a nucleic acid sequence using a cleavable adapter (see, e.g., FIG. 9). The method comprises fragmenting a nucleic acid sequence thereby producing a plurality of large nucleic acid sequence fragments of a specific size range. Onto each end of each nucleic acid sequence fragment a cleavable adapter is introduced (joined or ligated), wherein the cleavable adapter comprises an affinity tag and a cleavable linkage. The cleavable adapter is cleaved, thereby producing a plurality of nucleic acid sequence fragments having compatible adapter ends. The nucleic acid sequence fragments having compatible adapter ends are maintained under conditions in which the compatible adapter ends intramolecularly ligate, thereby producing a plurality of circularized nucleic acid sequences. The plurality of circularized nucleic acid sequences are fragmented, thereby producing a plurality of paired tags comprising a linked 5′ end tag and a 3′ end tag of each nucleic acid sequence fragment, wherein the 5′ end tag and 3′ end tag are joined by the intramolecularly ligated adapter ends. A paired tag library from a plurality of large nucleic acid sequence fragments is thereby produced. In one embodiment, the specific size range of the large nucleic acid fragments is from about 2 to about 200 kilobase pairs. In another embodiment, the large nucleic acid sequence fragments are produced by shearing. Sheared fragments can be blunt-ended and fractionated by agarose gel electrophoresis or pulsed field gel electrophoresis, as will be understood by a person of skill in the art. In a further embodiment, the plurality of circularized nucleic acid sequences are sheared to produce the plurality of paired tags comprising a 5′ end tag joined to a 3′ end tag of each nucleic acid sequence fragment by the intramolecularly ligated adapter ends. In a still further embodiment, the plurality of paired tags comprising a linked 5′ end tag and a 3′ end tag of each nucleic acid sequence fragment are blunt-ended. In another embodiment, the cleavable adapter further comprises a restriction endonuclease recognition site specific for a restriction endonuclease that cleaves a nucleic acid sequence distally to the restriction endonuclease recognition site. Thus, in one embodiment, the plurality of circularized nucleic acid can be cleaved by a restriction endonuclease that cleaves the nucleic acid sequence fragment distally to the restriction endonuclease recognition site.

In a further embodiment, the cleavable adapter comprises an affinity tag selected from the group consisting of biotin, digoxigenin, a hapten, a ligand, a peptide and a nucleic acid. In one embodiment, the plurality of paired tags comprising the linked 5′ end tag and a 3′ end tag of each nucleic acid sequence fragment are isolated by capturing the affinity tags, thereby producing an isolated paired tag library. In another embodiment, the method for producing a paired tag library from a nucleic acid sequence further comprises amplification of the isolated paired tag library to produce a library of amplified paired tags. Thus, in one embodiment, amplification comprises ligating a pair of asymmetrical adapters to the ends of each paired tag, wherein the pair of asymmetrical adapters comprise:

a first asymmetrical oligonucleotide adapter selected from the group consisting of:

(i) an asymmetrical tail adapter comprising a first ligatable end, and a second end comprising a single-stranded 3′ overhang of at least about 8 nucleotides;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region,
   and a second asymmetrical oligonucleotide adapter selected from the group consisting of:
(i) an asymmetrical tail adapter comprising a first ligatable end, and a second end comprising a single-stranded 5′ overhang of at least about 8 nucleotides, wherein the 3′ end of the strand that does not comprise the 5′ overhang comprises at least one blocking group;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region.
   In the method, the first and second asymmetrical oligonucleotide adapters are not identical. When the pair of asymmetrical adapters are ligated to the ends of each paired tag, a plurality of end-linked nucleic acid sequence fragments are produced, which is a library of end-linked paired tags. The library of end-linked paired tags are amplified in an amplification reaction. Thus, the method further comprises amplifying one strand of the each end-linked paired tag referred to herein as the template strand. The amplification reaction comprises (1) contacting the template strand with a first primer that is complementary to a first primer binding site in a first asymmetrical adapter in the template strand. Under appropriate conditions, the first primer synthesizes a first nucleic acid strand in the amplification reaction, wherein the first nucleic acid strand is complementary to the template strand, and wherein the 3′ end of the first nucleic acid strand comprises a second primer binding site that is complementary to a sequence in the second asymmetrical adapter in the template strand. The amplification reaction further comprises (2) contacting the first nucleic acid strand with a second primer that is complementary to the second primer binding site in the first nucleic acid strand under conditions in which a complementary strand of the first nucleic acid strand is synthesized. The amplification steps (1) and (2) are repeated, and amplifies the end-linked paired tags, thereby producing an amplified library of paired tags. In one embodiment, the amplified library of paired tags are characterized. In a particular embodiment, the amplified library of paired tags are sequenced. In a further embodiment, the method comprises sequencing the amplified library of paired tags. In another embodiment, the paired tag library is produced from a nucleic acid sequence that is a genome. In another embodiment, the cleavable linkage in the cleavable adapter is a 3′ phosphorothiolate linkage. Thus, in one embodiment, 3′ phosphorothiolate linkage is cleaved by Ag+, Hg2+ or Cu2+, at a pH of at least about 5 to at least about 9, and at a temperature of at least about 22° C. to at least about 37° C. In another embodiment, cleavable linkage in the cleavable adapter is a deoxyuridine nucleotide. Thus, in one embodiment, the deoxyuridine is cleaved by uracil DNA glycosylase (UDG) and an AP-lyase.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A is a schematic representation of a 3′ asymmetrical tail adapter and 5′ asymmetrical tail adapter, each having a double stranded region, ligated to a DNA fragment (“insert”). Numeral (1) represents a 3′ tail (or overhang) of the 3′ tail adapter; (2) represents the 5′ tail (or overhang) of the 5′ tail adapter; (5) represents a double-stranded region of the 3′ tail adapter or 5′ tail adapter; (7) represents ligatable ends of the 3′ tail adapter or 5′ tail adapter (see also FIG. 1D).

FIG. 1B is a schematic representation of two asymmetrical Y adapters, each having a double-stranded region, ligated to a DNA fragment (“insert”). Numerals (1), (2), (3), and (4) each represent single-stranded, non-complementary regions of the Y adapter (i.e., the “arms” of the Y adapter); (7) represents a ligatable end of the Y adapters (see also FIG. 1D).

FIG. 1C is a schematic representation of two asymmetrical bubble adapters, each having a double-stranded region, ligated to a DNA fragment (“insert”). Numerals (1), (2), (3), and (4) each represent single-stranded, non-complementary regions of the bubble adapters. Numerals (5) and (6) represent double-stranded regions of the bubble adapters; (7) represents a ligatable end of the bubble adapters (see also FIG. 1D).

FIG. 1D is a schematic representation of 3 different types of ligatable ends (7) of a double-stranded nucleic acid.

FIGS. 2(A-C) is a schematic representation of the possible amplification products that can be produced from a DNA fragment ligated to a 3′ Tail-adapter (A) and 5′ Tail-adapter (B). P1 and P2 represent primers for amplification.

FIGS. 3(A-C) is a schematic representation of the possible amplification products that can be produced from a DNA fragment ligated to a pair of different Y-adapters (A and B). P1 and P2 represent primers for amplification.

FIGS. 4(A-C) is a schematic representation of the possible amplification products that can be produced from a DNA fragment ligated to a pair of different bubble-adapters (A and B). P1 and P2 represent primers for amplification.

FIG. 5 is a photograph of agarose gel electrophoresis images demonstrating PCR amplification products corresponding in size to amplification products produced after ligation to a pair of asymmetrical linkers. Shown is a 4% agarose gel analysis of various asymmetric adapter ligation and PCR products. Lane 1: Invitrogen 10 bp ladder; Lanes 2,5: Adapters A and B were ligated and 1.25 fmol of the ligation product was used as template for a PCR reaction. Note that only the A-B product amplifies (not A-A, or B-B); Lanes 3,6: Same as lane 2 except 0.125 fmol of ligtion was used as template; Lane 4: same as lane 2 except 0.0125 fmol of ligtion was used as template; Lane 7: 0.0125 pmol of the AsymA and AsymB ligation was loaded to demonstrate that PCR in the previous lanes is responsible for the single band; Lane 8: no template PCR control; Lane 9: no primer PCR control with 0.00125 pmol template; Lane 10: 38 pmol of the adapter A+B ligation; Lane 11: Ligation of adapter A to itself; Lane 12: ligation of adapter A2 to itself; Lane 13: Ligation of adapter A+A2; Lane 14: Ligation of adapter A+B.

FIG. 6 is a schematic representation of a method for producing a paired end library using an affinity linker with MmeI or EcoP151 restriction endonuclease recognition sites.

FIGS. 7(A-B) is a photograph of agarose electrophoresis images showing purification of DNA fragments from different stages of genomic library preparation using the scheme illustrated in FIG. 6.

FIG. 8 is a photograph of agarose electrophoresis images showing PCR products produced from asymmetric linker primers from a genomic library prepared using the scheme illustrated in FIG. 6. Shown are PCR amplification products from an EcoP151 library (lanes 4 & 5) and MmeI Library (lanes 7 & 8). Lane 1 contains size markers correspond to an Invitrogen 25 bp ladder. The larger pair of bands for each library correspond to single-stranded and double-stranded amplification products (P) and the small bands indicated by the arrows correspond to linker dimers.

FIG. 9 is a schematic representation of a method for producing a paired end library using a cleavable adapter. An example of a cleavable adapter is also illustrated (SEQ ID NO: 23 [upper strand] and SEQ ID NO: 24 [lower strand]).

FIG. 10 is an outline of a method to make a 48 kb paired tag library using a COS-linker. The minimal lambda phage Cos site is shown (SEQ ID NO: 1). The recognition site for CosN and flanking sequence is also shown (SEQ ID NO: 2).

FIG. 11 is a schematic showing concatemers of COS linkers ligated to nucleic acid sequence fragments, and a graph depicting the expected size distribution for a genomic library packaged using cos-linkers and lambda packaging extracts.

FIG. 12 is an illustration of COS linker primers (CosP1 [SEQ ID NO: 3] and CosP2 [SEQ ID NO: 4]) comprising an EcoP151 restriction endonuclease recognition site which can be used to obtain a COS linker comprising an EcoP151 restriction endonuclease recognition site (SEQ ID NO: 5).

FIG. 13 is an illustration of COS linker primers (loxP1/lox71 [SEQ ID NO: 5] and loxP2/lox66 [SEQ ID NO: 6]) comprising loxP recombination sites which can be used to obtain a COS linker comprising loxP recombination sites (SEQ ID NO: 7).

FIG. 14 is a schematic outline for producing paired tags from a BAC clone library. As shown in the figure, in a particular embodiment, the asymmetrical adapters ligated to each end of the BAC paired ends are identical (represented as “AP1” and “1 PA” to illustrate the reverse orientations of the same adapter).

### DETAILED DESCRIPTION OF THE INVENTION

Sequencing of nucleic acid molecules derived from complex mixtures (e.g., mRNA populations) or entire genomes (e.g., a prokaryotic or eukaryotic genome) by a shotgun approach requires specific strategies for fragmenting and manipulating the starting nucleic acid molecules in order to facilitate accurate reconstruction of the sequences of those molecules. In the traditional whole genome sequencing strategy, the starting DNA is fragmented into smaller pieces in a variety of different size ranges (e.g., insert sizes of 2 kb, 10 kb, 40 kb and 150 kb) and cloned into vectors allowing replication and amplification in a bacterial host (e.g., high copy number plasmid, low copy number plasmid, fosmid and BAC vectors for propagation of the different insert sizes in E. coli). The cloned DNA fragments are purified and the two ends of each insert are sequenced from a large number of such clones (a sufficient number to represent the entire genome multiple times). Finally, the resulting paired-end sequences (each about 500-800 nucleotides in length) are subjected to computer based alignment and assembly to reconstruct the genome sequence. The use of a variety of different insert sizes enables the construction of a highly redundant, self consistent and self-confirming fragment scaffold based on the paired end sequences and known size distribution of the inserts in each size class, which ensures an accurate reconstruction of the starting sequence.

Although this approach has been successfully applied to many genomes, it invariably results in numerous gaps in the final reconstructed sequence after assembly at typical redundancy levels (e.g., 6-10× sequence coverage). This is caused by non-random sequence representation in the starting libraries resulting from loss of certain sequences during the shotgun cloning procedure, a phenomenon known as cloning bias. One source of such cloning bias results from the instability or low propagation efficiency of A:T-rich, G:C rich, repetitive (e.g. heterochromatin), palindromic or toxic coding sequences in multi-copy plasmids in E. coli. This results in the specific under-representation of such sequences in plasmid libraries, which has been observed in many bacterial, fungal, parasite, insect, plant and mammalian genome sequencing projects. The use of single-copy cloning vectors (e.g., fosmids and BACs) may reduce or eliminate some of those problems, but it is difficult to purify a sufficient amount of DNA from such vectors efficiently (e.g., in 384-well microplate format) and more expensive to sequence them than high copy number plasmids due to the requirement for larger amounts of expensive sequencing reagents.

Clone-based or hybrid approaches to whole genome sequencing utilizing collections of pre-mapped bacterial artificial chromosome (BAC) clones has been advocated as an alternative to the whole genome shotgun method, but is no longer considered a cost-effective alternative. This is due to the high cost and operational burden of producing genome-wide BAC maps, large numbers of individual BAC subclone libraries, the 15-20% waste associated with re-sequencing the BAC vector, the 5-20% waste associated with sequencing subclones derived from contaminating E. coli DNA in the BAC DNA preparations, the need to detect and remove transposon and bacteriophage insertions from the reconstructed BAC sequence, and the 20-50% waste in redundant sequencing of BAC overlaps.

Classical DNA sequencing techniques, such as the Maxam and Gilbert chemical cleavage method (Maxam and Gilbert, 1977, Proc. Natl. Acad. Sci. USA 74: 560-564; incorporated herein by reference) and the Sanger chain termination method (Sanger et al. 1977, Proc. Natl. Acad. Sci. USA 74: 5463-5467; incorporated herein by reference) are cumbersome and inefficient. Even with the advent of modified DNA polymerases, fluorescence energy transfer-based dideoxy terminator chemistry, highly efficient sample preparation automation and advanced fluorescence based capillary electrophoresis instruments (e.g., the ABI 3730xl), the throughput of the Sanger sequencing approach is still limited by the requirement for millions of individual template preparation and sequencing reactions to be produced in order to derive the nucleotide sequence of an entire genome.

Several alternative sequencing approaches that utilize massively parallel amplification or surfaces or on individual microbeads from millions of molecules in a single reaction vessel have been described in recent years. Examples include the Church polony technology (Mitra et al., 2003, Analytical Biochemistry 320, 55-65; Shendure et al., 2005 Science 309, 1728-1732; U.S. Pat. No. 6,432,360, U.S. Pat. No. 6,485,944, U.S. Pat. No. 6,511,803) the 454 picotiter pyrosequencing technology (Margulies et al., 2005 Nature 437, 376-380; US 20050130173), the Solexa single base addition technology (Bennett et al., 2005, Pharmacogenomics, 6, 373-382; U.S. Pat. No. 6,787,308, U.S. Pat. No. 6,833,246), the Lynx massively parallel signature sequencing technology (Brenner et al. (2000). Nat Biotechnol 18, 630-634; U.S. Pat. No. 5,695,934, U.S. Pat. No. 5,714,330) and the Adessi PCR colony technology (Adessi et al. (2000). Nucleic Acids Res 28, E87; WO00018957). All of these methods, as currently practiced, rely on PCR based template generation procedures. Although it is possible to produce short fragments suitable for PCR amplification and paired end sequence generation, efficient methods for doing so from long DNA fragments have not been described.

Thus, a pressing need exists for alternatives to conventional cloning procedures for generating paired-end sequences from genomic or mRNA derived fragments. Ideally, such alternatives would enable the construction of truly random fragment libraries in a wide range of size classes (e.g., 2 kb, 5 kb, 10 kb, 50 kb, 100 kb or 200 kb with a narrow window of size variation within each class) in a suitable format for DNA sequencing and without any prior passage through a bacterial host. The randomness of fragment end points is critical to complete genome assembly without gaps. Libraries produced by means of fragmentation with restriction endonucleases, which have been disclosed previously (e.g., in U.S. Pat. No. 6,054,276, U.S. Pat. No. 6,720,179 and WO03/074734), are not sufficiently random because the occurrence of restriction endonuclease cleavage sites is sparse, sequence dependent, highly variable and non-random in nature. An ideal method would also provide a reliable means to amplify genomic DNA fragments with high fidelity by PCR, for example, in such a way as to ensure that each amplified fragment ends up with a different universal primer sequence at each end. This is desirable because a variety of the new, potentially very inexpensive sequencing technologies that utilize massively parallel amplification on beads or surfaces from millions of molecules in a single experiment utilize a template generation strategy that requires a different universal priming site at each end of the starting DNA fragments. In addition, it would be useful for such a method to allow amplification of a single strand from a double-stranded nucleic acid sequence to facilitate heterozygosity analysis or characterization of hemi-methylation status. Thus, the present invention provides compositions and methods to achieve those ends, as well as providing methods useful for whole genome SNP discovery, genotyping, karyotyping, and characterization of insertions, deletions, inversions, translocations and copy number polymorphisms.

The present invention provides asymmetrical oligonucleotide adapters which can be used for the exponential amplification of a nucleic acid sequence wherein the resulting amplified product will have a different nucleic acid sequence on each end. In addition, the asymmetrical adapters permit the exponential amplification of a single strand from a double-stranded nucleic acid sequence. The present invention also provides methods for the generation of paired end libraries of DNA fragments wherein the paired ends are derived from the ends of DNA molecules about 2-200 kb in size.

As used herein, an asymmetrical adapter can comprise a ligatable end and at least one unpaired or single-stranded region wherein the nucleic acid sequence of one strand is not complementary to the nucleic acid sequence of the other strand. The unpaired region can be of any appropriate size, for example, from at least about 3 nucleotides to at least about 200 nucleotides, at least about 4 nucleotides to at least about 150 nucleotides, at least about 5 nucleotides to at least about 100 nucleotides, at least about 2 nucleotides to at least about 20 nucleotides, at least about 3 nucleotides to at least about 10 nucleotides, at least about 5 nucleotides to at least about 7 nucleotides, at least about 5 nucleotides to at least about 25 nucleotides, at least about 5 nucleotides to at least about 50 nucleotides, at least about 20 nucleotides to at least about 100 nucleotides, or longer, as will be appreciated by a person of skill in the art. In one embodiment, the length of the unpaired region is sufficient to permit primer binding for amplification, wherein at least the 3′ region of the primer can bind to the unpaired region of the asymmetrical linker or adapter.

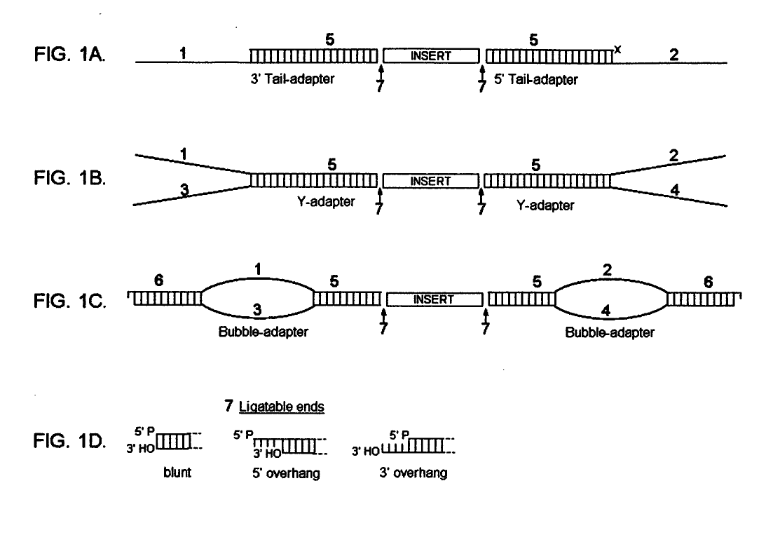

As used herein, a single-stranded region, tail, or overhang, is a single-stranded nucleic acid sequence extension at either end (e.g., 5′ end; 3′ end) of an asymmetrical oligonucleotide tail adapter (linker), in which the longer strand of the asymmetrical tail adapter is not base paired with a reverse complementary sequence in the other (opposite) strand (see, e.g., FIG. 1A), as will be understood by one of skill in the art. In one embodiment, the 3′ overhang of the first asymmetrical double-stranded oligonucleotide adapter and/or the 5′ overhang of the second asymmetric double-stranded oligonucleotide adapter are each at least about 8 nucleotides to at least about 100 nucleotides, at least about 3 nucleotides to at least about 200 nucleotides, at least about 4 nucleotides to at least about 150 nucleotides, at least about 5 nucleotides to at least about 100 nucleotides, at least about 15 nucleotides to at least about 90 nucleotides, at least about 20 nucleotides to at least about 75 nucleotides, at least about 2 nucleotides to at least about 20 nucleotides, at least about 4 nucleotides to at least about 10 nucleotides, at least about 6 nucleotides to at least about 9 nucleotides, at least about 5 nucleotides to at least about 25 nucleotides, at least about 5 nucleotides to at least about 50 nucleotides, at least about 20 nucleotides to at least about 100 nucleotides, or longer in length. In another embodiment, the 3′ overhang of the first asymmetrical double-stranded oligonucleotide adapter and the 5′ overhang of the second asymmetric double-stranded oligonucleotide adapter are each at least about 25 nucleotides to at least about 50 nucleotides, at least about 30 nucleotides to at least about 40 nucleotides in length. In one embodiment, the overhang in the first and second asymmetrical tail adapters are identical in length. In another embodiment, the overhang in the first and second asymmetrical tail adapters are different in length. In a further embodiment, the 3′ overhang of the first asymmetrical double-stranded oligonucleotide adapter comprises at least one primer binding site.

As described herein, the double-stranded oligonucleotide adapter can comprise at least one blocking group. As used herein, a blocking group is an agent or substituent that prevents nucleic acid sequence extension (e.g., by DNA polymerase or DNA ligase) and hence also prevents amplification of a nucleic acid sequence comprising the blocking group. Examples of 3′ blocking groups which may be present on a terminal 2′ deoxynucleotide include 3′ deoxy, 3′ phosphate, 3′ amino, or 3′-O—R nucleotide where R represents an alkyl, allyl, aryl or heterocyclic substituent. In a particular embodiment, the second asymmetrical tail adapter comprises a blocking group.

As used herein, “double stranded” refers to a paired nucleic acid sequence, wherein the two strands are substantially complementary to each other such that the two strands can form a paired structure (e.g., a double helix). As will be understood by the person of skill in the art, the two strands may contain one or more mismatches still retain a paired structure. In a particular embodiment, the paired structure is stable.

As described herein, an asymmetrical adapter can comprise a ligatable end. As used herein, a ligatable end is a sequence in a double-stranded oligonucleotide that has either a blunt end or a sticky-end. As will be understood by one of skill in the art, a blunt end has no 5′ or 3′ overhang in a double stranded nucleic acid molecule and a sticky end has either a 5′ or a 3′ overhang. Both blunt ends and sticky ends can be ligated to another compatible end. As used herein, a compatible end is a blunt end that can ligate with another blunt-ended nucleic acid sequence, or a sticky end comprising an overhang which can ligate with another sticky end that comprises essentially the reverse complementary overhang. Thus, sticky ends permit sequence-dependent ligation, whereas blunt ends permit sequence-independent ligation. Compatible ends and, thus, ligatable ends are produced by any known methods that are standard in the art. For example, compatible ends of a nucleic acid sequence are produced by restriction endonuclease digestion of the 5′ and/or 3′ end. In another embodiment, compatible ends of a nucleic acid sequence are produced by introducing (for example, by annealing, ligating, or recombining) an adapter to the 5′ end and/or 3′ end of the nucleic acid sequence, wherein the adapter comprises a compatible end, or alternatively, the adapter comprises a recognition site for a restriction endonuclease that produces a compatible end on cleavage. Blunt ends can be produced by digestion with a site-specific endonuclease (e.g., a restriction endonuclease), a non-specific double-standed DNA specific endonuclease (e.g., DNA polymerase I in the presence of Mn²⁺) or by random shearing (e.g., by sonication, acoustic energy, or hydrodynamic shearing by forcing a DNA solution through a small orifice under pressure). After random shearing or DNAase digestion the DNA ends are often frayed (contain short 5′ or 3′ overhangs with or without terminal phosphate groups). The frayed ends are converted to ligatable ends by blunt-ending, or healing, using one or more of the following: a DNA polymerase, a mixture of dATP, dCTP, dGTP and dTTP, a DNA polymerase having strong 3′ to 5′ and 5′ to 3′ exonuclease activities, polynucleotide kinase, ATP, a single stranded DNA specific exonuclease, a single stranded DNA specific endonuclease.

The asymmetrical adapters of the present invention can also comprise, or be used in conjunction with affinity linkers. The affinity linker can be ligated, for example, between two nucleic acid sequences, thereby linking the two nucleic acid sequences. As used herein, an affinity linker comprises two ligatable ends and at least one affinity tag. Either or both of the ligatable ends can be ligated to a nucleic acid sequence. In one embodiment, both ligatable ends of the affinity linker can be ligated to either end of one nucleic acid sequence, thereby circularizing the nucleic acid sequence. In another embodiment, each ligatable end of the affinity linker can be ligated to different nucleic acid sequences, thereby producing a concatemer of the different nucleic acid sequences. As used herein, an affinity tag is an agent that can be used to purify, select, identify, locate and/or enrich for molecules comprising the affinity tag. For example, an affinity tag can be biotin, digoxigenin, a hapten, a ligand, a peptide and/or a nucleic acid. An affinity linker can comprise multiple affinity tags that are the same or different. An affinity linker of the present invention is at least about 15 nucleotides to about 100 nucleotides, at least about 25 nucleotides to about 75 nucleotides, or at least about 35 nucleotides to about 60 nucleotides. The affinity linker therefore provides for purification, isolation, selection, location, enrichment or identification affinity-linked nucleic acid sequences.

An asymmetrical adapter of the present invention can also comprise a primer binding site. As used herein, a primer binding site can comprise a sequence that binds a whole primer length, or the primer binding site can comprise a sequence that binds to a sufficient portion of the 3′ end of the primer, wherein the portion is sufficient to permit primer binding, e.g., for primer extension and/or amplification. In one embodiment, the single-stranded overhang of the first asymmetrical oligonucleotide tail adapter comprises at least one primer binding site. In another embodiment, the unpaired region of a Y adapter or a bubble adapter comprises at least one primer binding site.

As described herein, the asymmetrical adapters of the present invention can be used for amplification of one or more nucleic acid molecules. As used herein, amplification or an amplification reaction refers to methods for amplification of a nucleic acid sequence including polymerase chain reaction (PCR), ligase chain reaction (LCR), rolling circle amplification (RCA), and strand displacement amplification (SDA), as will be understood by a person of skill in the art. Such methods for amplification comprise e.g., primers that anneal to the nucleic acid sequence to be amplified, a DNA polymerase, and nucleotides. Furthermore, amplification methods, such as PCR, can be solid-phase amplification, polony amplification, colony amplification, emulsion PCR, bead RCA, surface RCA, surface SDA, etc., as will be recognized by one of skill in the art. In addition, it will be recognized that it is advantageous to utilize amplification protocols that maximize the fidelity of the amplified products to be used as templates in DNA sequencing procedures. Such protocols utilize, for example, DNA polymerases with strong discrimination against misincorporating incorrect nucleotides and/or strong 3′ exonuclease activities (also referred to as proofreading or editing activities) to remove misincorporated nucleotides during polymerization.

Nucleic acid sequences that can be amplified include e.g., DNA, a genome, a fragment of a genome, a chromosome, a molecularly cloned DNA molecule, e.g., a BAC, etc.

In one embodiment of the present invention, the pair of asymmetrical adapters are not identical. As used herein, two (or more) asymmetrical adapters are “non-identical” or “not identical” when the asymmetrical adapters differ from each other by at least one nucleotide in a primer binding site, by at least one nucleotide in the complementary nucleic acid sequence of a primer binding, and/or by the presence or absence of a blocking group. Furthermore, the two (or more) non-identical asymmetrical adapters can have substantial differences in nucleic acid sequences. For example, two asymmetrical tail adapters, asymmetrical bubble adapters or two asymmetrical Y adapters (described in more detail below) can comprise entirely different sequences (e.g., with little or no sequence identity). In a particular embodiment, the non-identical asymmetrical adapters have little or no sequence identity in the unpaired region (e.g., the tail region, the arms of the Y region, or the bubble region). Alternatively, a pair of asymmetrical adapters are not identical such that they differ in kind or type, e.g., the first and second asymmetrical adapters are not both asymmetrical tail adapters, not both asymmetrical Y adapters, or not both asymmetrical bubble adapters. That is, a pair of asymmetrical adapters can comprise, e.g., an asymmetrical tail adapter and a bubble adapter or Y adapter, or a pair of asymmetrical adapters can comprise a bubble and a Y adapter. In a particular embodiment, two (or more) asymmetrical adapters that are not identical in kind or type differ from each other by at least one nucleotide in a primer binding site, by at least one nucleotide in the complementary nucleic acid sequence of a primer binding, and/or by the presence or absence of a blocking group.

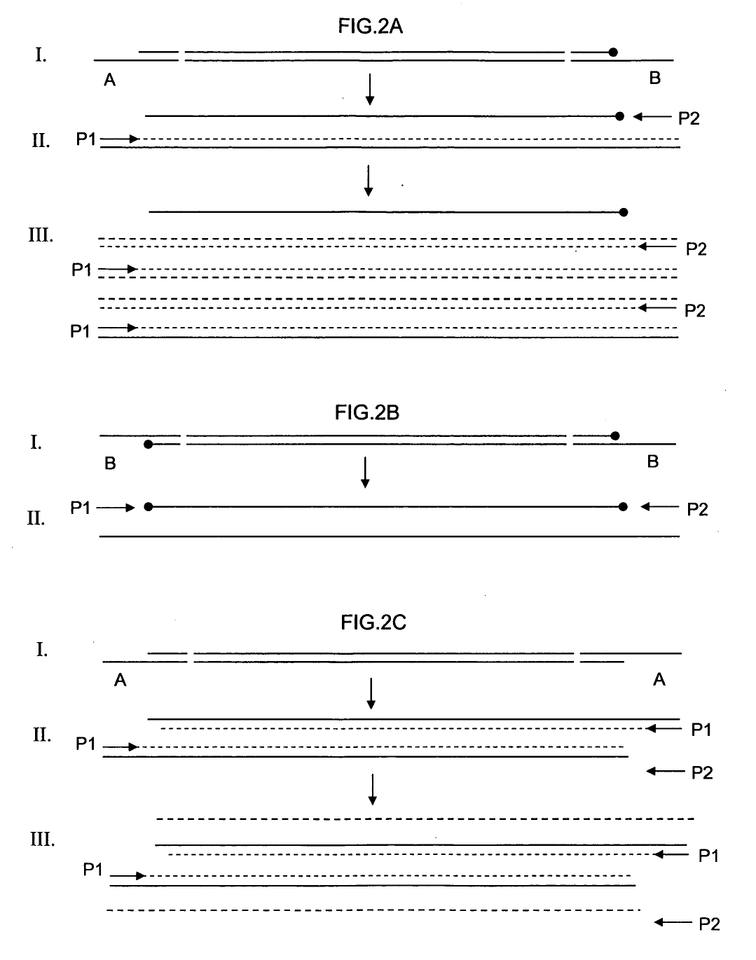

In one embodiment a pair of asymmetrical adapters may comprise a pair of tail oligonucleotide adapters (also referred to herein as tail adapters, 3′ tail adapter and 5′ tail adapter, asymmetrical tail adapters, asymmetrical oligonucleotide adapters, asymmetrical adapters, “JamAdapters”, “JamLinkers” and variations thereof), see, e.g., FIGS. 1A-C. A pair of tail adapters comprises: (a) a first partially double-stranded oligonucleotide adapter which comprises one ligatable end and a 3′ single-stranded tail (or overhang) at the opposite end; and (b) a second partially double-stranded oligonucleotide adapter which comprises one ligatable end, a 5′ single-stranded tail (or overhang) a the opposite end with at least one blocking group at the 3′ end of the strand that does not comprise the 5′ overhang, wherein the first and second tail adapters are not identical. In one embodiment, the 3′ tail of the first asymmetrical oligonucleotide adapter and the 5′ tail of the second asymmetrical oligonucleotide adapter are each at least about 8 nucleotides to at least about 100 nucleotides, at least about 15 nucleotides to at least about 90 nucleotides, or at least about 20 nucleotides to at least about 75 nucleotides in length. In another embodiment, the 3′ tail of the first asymmetrical oligonucleotide adapter and the 5′ tail of the second asymmetrical oligonucleotide adapter are each at least about 25 nucleotides to at least about 50 nucleotides, at least about 30 nucleotides to at least about 40 nucleotides in length. In a further embodiment, the 3′ tail of the first asymmetrical oligonucleotide adapter comprises at least one primer binding site. The primer binding site permits, e.g., amplification of a nucleic acid molecule that is ligated to the pair of asymmetrical adapters. In a particular embodiment, the pair of asymmetrical tail adapters permits the amplification of one strand in a double-stranded nucleic acid molecule that is ligated to the pair of asymmetrical adapters (see, e.g., FIG. 2). As described herein, the second asymmetrical tail adapter can comprise at least one blocking group. The blocking group prevents e.g., sequence extension in an amplification reaction, as will be understood by a person of skill in the art.

In another embodiment, a pair of asymmetrical adapters may comprise a pair of Y oligonucleotide adapters (also referred to herein as Y adapters, asymmetrical Y adapters, asymmetrical adapters or asymmetrical oligonucleotide adapters). See, e.g., FIG. 1B. A pair of asymmetrical Y oligonucleotide adapters comprise: (a) a first partially double-stranded Y oligonucleotide adapter comprising a first paired, ligatable end, and a second unpaired end which comprises two non-complementary strands; and (b) a second partially double-stranded Y oligonucleotide adapter comprising a first paired, ligatable end, and a second unpaired end which comprises two non-complementary strands, wherein the first and second asymmetrical Y oligonucleotide adapters are not identical. In one embodiment, the length of the non-complementary strands in either or both of the first or second Y oligonucleotide adapter are at least about 8 nucleotides in length. In another embodiment, the non-complementary strands are at least about 8 nucleotides to at least about 100 nucleotides in length. In another embodiment, the non-complementary strands are at least about 25 nucleotides to at least about 40 nucleotides in length. The length of the non-complementary strands in each Y adapter can be the same or different. In one embodiment, at least one non-complementary strand of the first (or second) Y adapter comprises at least one primer binding site. In a particular embodiment, one or both tails in the asymmetrical Y oligonucleotide adapter comprise a sufficient region of single-stranded nucleic acid sequence for primer binding.

In another embodiment, a pair of asymmetrical adapters may comprise a pair of bubble oligonucleotide adapters (also referred to herein as bubble adapters, asymmetrical bubble adapters, asymmetrical adapters or asymmetrical oligonucleotide adapters). See, e.g., FIG. 1C. A pair of asymmetrical bubble oligonucleotide adapters comprise: (a) a first partially double-stranded bubble oligonucleotide adapter comprising at least one unpaired region flanked on each side by a paired region; and (b) a second asymmetrical bubble oligonucleotide adapter comprising at least one unpaired region flanked on each side by a paired region, wherein the first and second asymmetrical bubble oligonucleotide adapters are not identical. In one embodiment, the unpaired region in the bubble adapter is at least about 8 nucleotides in length. In another embodiment, the unpaired region in a bubble adapter is at least about 5 to about 25 nucleotides in length. In another embodiment, the unpaired region in a bubble adapter is at least about 8 to at least about 15 nucleotides in length. In a particular embodiment, a bubble adapter comprises more than one unpaired region. In one embodiment, the unpaired region in the first bubble adapter comprises at least one primer binding site. In a particular embodiment, the unpaired region in the asymmetrical bubble oligonucleotide adapter comprises a sufficient region of single-stranded nucleic acid sequence for primer binding.

In another embodiment of the invention, a pair of asymmetrical oligonucleotide adapters (e.g., for amplification of at least one double stranded nucleic acid molecule, wherein the amplification produces a plurality of amplified nucleic acid molecules having a different nucleic acid sequence at each end), comprises a pair of adapters wherein the first and second asymmetrical oligonucleotide adapters are not identical. For example, the pair of asymmetrical oligonucleotide adapters are two different adapters selected from the group consisting of: an asymmetrical oligonucleotide adapter comprising a first ligatable end, and a second end comprising a single-stranded 3′ overhang of at least about 8 nucleotides; an asymmetrical oligonucleotide adapter comprising a first ligatable end, and a second end with a single-stranded 5′ overhang comprising at least about 8 nucleotides, wherein the 3′ end of the strand that does not comprise the 5′ overhang comprises at least one blocking group; an asymmetrical Y oligonucleotide adapter comprising a first ligatable end, and a second unpaired end comprising two single-stranded tails, wherein the length of the single-stranded regions are at least about 8 nucleotides; and an asymmetrical bubble oligonucleotide adapter comprising an unpaired region of at least about 8 nucleotides flanked on each side by a paired region.

The asymmetrical adapters of the present invention can be used in a variety of ways, such as for amplification of a nucleic acid molecule. In one aspect of the invention, provided herein is a method for amplification of at least one double-stranded nucleic acid molecule to produce a plurality of amplified molecules having a different sequence at each end. The presence of a different sequence at either end of an amplified molecule permits, e.g., the identification of the beginning and end of a nucleic acid molecule when multiple nucleic acid molecules are present in a concatemer. The method also provides for the selective amplification of a single strand of a nucleic acid sequence. The selective amplification of one strand (also referred to herein as a template strand) of a double-stranded nucleic acid molecule that is ligated to a pair of asymmetrical adapters (referred to herein as an end-linked nucleic acid molecule and variations thereof, wherein one asymmetrical adapter is ligated to one end of the nucleic acid molecule, e.g., the 5′ end or “left” side of the nucleic acid molecule, and a second asymmetrical adapter is ligated to the other end of the nucleic acid molecule, e.g., the 3′ end or “right” side) is achieved by designing appropriate primers to bind to only nucleic acid sequences on the template strand (see, e.g., FIGS. 2-4). The template strand can be either the “upper” strand (e.g., sense or coding strand) or “lower” strand (e.g., anti-sense or reverse complementary strand of the coding strand) of a double-stranded nucleic acid molecule.

In one embodiment, an end-linked nucleic acid molecule, wherein the end-linked nucleic acid molecule comprises one strand of the end-linked nucleic acid molecule referred to herein as the template strand, is amplified. The amplification reaction comprises (1) contacting the template strand with a first primer that is complementary to a first primer binding site in a first asymmetrical adapter in the template strand. Under appropriate conditions, the first primer synthesizes a first nucleic acid strand in the amplification reaction, wherein the first nucleic acid strand is complementary to the template strand, and wherein the 3′ end of the first nucleic acid strand comprises a second primer binding site that is complementary to a sequence in the second asymmetrical adapter in the template strand. The amplification reaction further comprises (2) contacting the first nucleic acid strand with a second primer that is complementary to the second primer binding site in the first nucleic acid strand under conditions in which a complementary strand of the first nucleic acid strand is synthesized. The amplification steps (1) and (2) are repeated, thereby exponentially amplifying the template strand.

In a particular embodiment of the invention, the method for amplification of at least one double-stranded nucleic acid molecule comprises ligating to one end of the double-stranded nucleic acid molecule a first asymmetrical oligonucleotide adapter selected from the group consisting of:
(i) an asymmetrical oligonucleotide adapter comprising a first ligatable end, and a second end comprising a single-stranded 3′ overhang of at least about 8 nucleotides;
(ii) an asymmetrical Y oligonucleotide adapter comprising a first ligatable end, and a second unpaired end comprising two single-stranded tails, wherein the length of the single-stranded tails are at least about 8 nucleotides; and
(iii) an asymmetrical bubble oligonucleotide adapter comprising an unpaired region of at least about 8 nucleotides flanked on each side by a paired region.
   The method further comprises ligating to the other end of the double-stranded nucleic acid molecule a second asymmetrical oligonucleotide adapter selected from the group consisting of:
(i) an asymmetrical oligonucleotide adapter comprising a first ligatable end, and a second end with a single-stranded 5′ overhang comprising at least about 8 nucleotides, wherein the 3′ end of the strand that does not comprise the 5′ overhang comprises at least one blocking group;
(ii) an asymmetrical Y oligonucleotide adapter comprising a first ligatable end, and a second unpaired end comprising two single-stranded tails, wherein the length of the single-stranded tails are at least about 8 nucleotides; and
(iii) an asymmetrical bubble oligonucleotide adapter comprising an unpaired region of at least about 8 nucleotides flanked on each side by a paired region,
   wherein the first and second asymmetrical oligonucleotide adapters are not identical, thereby producing an end-linked double-stranded nucleic acid molecule. The method further comprises amplifying one strand of the end-linked nucleic acid molecule referred to herein as the template strand. The amplification reaction comprises (1) contacting the template strand with a first primer that is complementary to a first primer binding site in a first asymmetrical adapter in the template strand. Under appropriate conditions, the first primer synthesizes a first nucleic acid strand in the amplification reaction, wherein the first nucleic acid strand is complementary to the template strand, and wherein the 3′ end of the first nucleic acid strand comprises a second primer binding site that is complementary to a sequence in the second asymmetrical adapter in the template strand. The amplification reaction further comprises (2) contacting the first nucleic acid strand with a second primer that is complementary to the second primer binding site in the first nucleic acid strand under conditions in which a complementary strand of the first nucleic acid strand is synthesized. The amplification steps (1) and (2) are repeated, and the amplification produces a plurality of amplified molecules from the template strand, wherein the plurality of amplified molecules each have a different sequence at each end. As already noted, a primer binding site can comprise a sequence that binds a whole primer length, or the primer binding site can comprise a sequence that binds to a sufficient portion of the 3′ end of the primer, wherein the portion is sufficient to permit primer binding for amplification.

In one embodiment, the method for amplification is exponential amplification (versus linear amplification) of one strand in a double-stranded nucleic acid molecule.

In a further aspect of the invention, provided herein is a method for producing and amplifying a paired tag from a first nucleic acid sequence fragment, without cloning. As used herein, a “paired tag” (also referred to herein as a “paired end”) is a nucleic acid sequence comprising a 5′ end of a contiguous nucleic acid sequence paired or joined with the 3′ end of the same contiguous nucleic acid sequence, wherein a portion of the internal sequence of the contiguous nucleic acid sequence is removed. Paired tags are also described in U.S. patent application Ser. No. 10/978,224, the teachings of which are herein incorporated by reference in their entirety. The 5′ end and 3′ end can be paired or joined by a variety of methods known to those of skill in the art. For example, the 5′ end and 3′ end can be paired or joined directly by ligation, chemical crosslinking and the like, or indirectly by via an adapter or a linker. In one embodiment, a paired tag can be represented as:
5′- - - - - -▪- - - - - -3′
   wherein “5′- - - - - -” represents a 5′ end tag, of a contiguous sequence, “- - - - - -3′” represents a 3′ end tag of the same contiguous sequence, and “▪” represents a linker (or adapter) that links the 5′ end tag to the 3′ end tag.

Alternatively, a paired tag can be represented as:
- - - - - -50′▪3′- - - - - -
   wherein “- - - - - -5′” represents a 5′ end tag, “3′- - - - - -” represents a 3′ end tag, and “▪” represents an adapter or linker. In this embodiment, the 5′ end tag and 3′ end tag are joined to each other via a linker or adapter in opposite orientation to that in the original nucleic acid sequence.

Still further, a paired tag can be represented as:
▪- - - - - -5′▪ 3′- - - - - -▪
   wherein “- - - - - -5′” represents a 5′ end tag, “3- - - - - -” represents a 3′ end tag, and “▪” represents an adapter or linker. The adaptors or linkers as illustrated can be either the same or different. As will be also recognized by the person of skill in the art, the orientation of the 5′ end tag and 3′ end tag can be reversed. As discussed below, the linker or adapter can comprise: at least one endonuclease recognition site, (e.g., for a restriction endonuclease enzyme such as a rare cutting enzyme, an enzyme that cleaves distally to its recognition sequence); an overhang that is compatible with joining to a complementary overhang from a restriction endonuclease digestion product; an attachment capture moiety, such as biotin; primer sites (for use in, e.g., amplification, RNA polymerase reactions); Kozak sequence, promoter sequence, (e.g. T7 or SP6); and/or an identifying moiety, such as a fluorescent label.

A paired tag is distinguished from a ditag since a ditag is a randomized pairing of two tags usually from more than one nucleic acid sequence (e.g., a 5′ end of sequence A and the 3′ end of sequence B or a 5′ end of sequence A and the 5′ end of sequence B, wherein sequence A and B are non-contiguous). In contrast, a paired tag as described herein, is not a randomized pairing of two tags, but the pairing of two tags that are produced from the ends of a single contiguous nucleic acid sequence.

Paired tags facilitate the assembly (such as whole genome assembly, or genome mapping) of a nucleic acid sequence, such as a genomic DNA sequence, even if either tag (for example, the 5′ tag) is generated from a non-informative sequence (for example, a repeat sequence) and the other tag in the pair (for example, the 3′ tag) is generated from an informative sequence based on the paired tag's “signature”. A paired tag's signature is derived from the size of the original nucleic acid sequence from which the paired tag represents the 5′ end and 3‘end of the paired tag’s nucleic acid sequence. The random association of tags to form ditags does not retain any signature as the two tags in the ditag generally do not represent the 5′ end and 3′ end of any contiguous nucleic acid sequence. In addition, a paired tag can identify the presence of an inverted nucleic acid sequence in, for example, a genomic DNA sample, because of the paired tag's signature. Randomly associated tags that form ditags cannot detect the presence of an inverted nucleic acid sequence because the ditag does not retain a signature. For example, a database version of one genome places tags in the order of: X-Y-Z-A in a contiguous sequence. Paired tags from this sequence generates the following three paired tags: X-Y, Y-Z and Z-A. In a comparison genome, for example, from a cancer cell, the paired tags from the same contiguous sequence generate the following three paired tags: X-Z, Z-Y and Y-A. The presence of the latter three paired tags indicates that the order of the tags in the contiguous sequence of the cancer cell genome is: X-Z-Y-A. Thus, it is determined that the fragment Y-Z is inverted. Ditags will not have sufficient information to determine if a contiguous sequence has an inversion due to the random association of any two tags together.

A “5′ end tag” (also referred to as a “5′ tag”) and a “3′ end tag” (also referred to as a “3′ tag”) of a contiguous nucleic acid sequence can be short nucleic acid sequences, for example, the 5′ end tag or 3′ end tag can be from about 6 to about 80 nucleotides, from about 6 to about 600 nucleotides, from about 6 to about 1200 nucleotides or longer, from about 10 to about 80 nucleotides, from about 10 to about 1200 nucleotides, from about 10 to about 1500 nucleotides or longer in length that are from the 5′ end and 3′ end, respectively, of the contiguous nucleic acid sequence. In one embodiment, the 5′ end tag and/or the 3′ end tag are about 14 nucleotides, about 20 nucleotides or about 27 nucleotides. The 5′ end tag and a 3′ end tag are generally sufficient in length to identify the contiguous nucleic acid sequence from which they were produced. In one embodiment, the 5′ end tag and/or the 3′ end tag are produced after cleavage of the contiguous nucleic acid sequence with a restriction endonuclease having a recognition site located at the 5′ and/or 3′ end of the contiguous nucleic acid sequence. In a particular embodiment, the restriction endonuclease cleaves the contiguous nucleic acid sequence distally to (outside of) its restriction endonuclease recognition site. The 5′ end tag and/or 3′ end tag can also be produced after cleavage by other fragmentation means, such as random shearing, treatment with non-specific endonucleases or other fragmentation methods as will be understood by one skilled in the art. In some embodiments, cleavage can occur in a linker or adapter sequence, in other embodiments, cleavage can occur outside a linker or adapter sequence, such as in a genomic DNA fragment.

One method for producing and amplifying a paired tag comprises joining the 5′ and 3′ ends of a first nucleic acid sequence fragment via a first linker such that the first linker is located between the 5′ end and the 3′ end of the first nucleic acid sequence fragment in a circular nucleic acid molecule. The circular nucleic acid molecule is cleaved, thereby producing a second nucleic acid sequence fragment, wherein a 5′ end tag of the first nucleic acid sequence fragment is joined to a 3′ end tag of the first nucleic acid sequence fragment via the first linker. A pair of asymmetrical second adapters are ligated to the ends of the second nucleic acid sequence fragment, wherein the pair of asymmetrical adapters comprise:

a first asymmetrical oligonucleotide adapter selected from the group consisting of:

(i) an asymmetrical tail adapter comprising a first ligatable end, and a second end comprising a single-stranded 3′ overhang of at least about 8 nucleotides;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region,
   and a second asymmetrical oligonucleotide adapter selected from the group consisting of:
(i) an asymmetrical tail adapter comprising a first ligatable end, and a second end comprising a single-stranded 5′ overhang of at least about 8 nucleotides, wherein the 3′ end of the strand that does not comprise the 5′ overhang comprises at least one blocking group;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region.
   In the method, the first and second asymmetrical oligonucleotide adapters are not identical. When the pair of asymmetrical adapters are ligated to the ends of the second nucleic acid sequence fragment, an end-linked nucleic acid sequence fragment is produced. The method further comprises amplifying one strand of the end-linked nucleic acid molecule referred to herein as the template strand. The amplification reaction comprises (1) contacting the template strand with a first primer that is complementary to a first primer binding site in a first asymmetrical adapter in the template strand. Under appropriate conditions, the first primer synthesizes a first nucleic acid strand in the amplification reaction, wherein the first nucleic acid strand is complementary to the template strand, and wherein the 3′ end of the first nucleic acid strand comprises a second primer binding site that is complementary to a sequence in the second asymmetrical adapter in the template strand. The amplification reaction further comprises (2) contacting the first nucleic acid strand with a second primer that is complementary to the second primer binding site in the first nucleic acid strand under conditions in which a complementary strand of the first nucleic acid strand is synthesized. The amplification steps (1) and (2) are repeated, and the amplification produces a plurality of amplified molecules from the template strand, wherein the plurality of amplified molecules each have a different sequence at each end. As a result, a paired tag from a first nucleic acid sequence fragment is produced and amplified without cloning (i.e., without passage through live E. coli cells).

In a still further aspect of the invention, provided herein is a method for characterizing a nucleic acid sequence, without cloning. The method for characterizing a nucleic acid sequence, without cloning comprises fragmenting a nucleic acid sequence thereby producing a plurality of first nucleic acid sequence fragments having a 5′ end and a 3′ end, joining the 5′ and 3′ ends of each first nucleic acid sequence fragment to a first linker such that the first linker is located between the 5′ end and the 3′ end of each first nucleic acid sequence fragment in a circular nucleic acid molecule, cleaving the circular nucleic acid molecules, thereby producing a plurality of second nucleic acid sequence fragments wherein a subset of the fragments comprise a paired tag derived from each first nucleic acid sequence fragment joined via the first linker, ligating a pair of asymmetrical second adapters to the ends of the second nucleic acid sequence fragment, wherein the pair of asymmetrical adapters comprise:

a first asymmetrical oligonucleotide adapter selected from the group consisting of:

(i) an asymmetrical tail adapter comprising a first ligatable end, and a second end comprising a single-stranded 3′ overhang of at least about 8 nucleotides;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region,
   and a second asymmetrical oligonucleotide adapter selected from the group consisting of:
(i) an asymmetrical tail adapter comprising a first ligatable end, and a second end comprising a single-stranded 5′ overhang of at least about 8 nucleotides, wherein the 3′ end of the strand that does not comprise the 5′ overhang comprises at least one blocking group;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region.
   In the method, the first and second asymmetrical oligonucleotide adapters are not identical. When the pair of asymmetrical adapters are ligated to the ends of the second nucleic acid sequence fragment, an end-linked nucleic acid sequence fragment is produced. The method further comprises amplifying one strand of the end-linked nucleic acid molecule referred to herein as the template strand The amplification reaction comprises (1) contacting the template strand with a first primer that is complementary to a first primer binding site in a first asymmetrical adapter in the template strand. Under appropriate conditions, the first primer synthesizes a first nucleic acid strand in the amplification reaction, wherein the first nucleic acid strand is complementary to the template strand, and wherein the 3′ end of the first nucleic acid strand comprises a second primer binding site that is complementary to a sequence in the second asymmetrical adapter in the template strand. The amplification reaction further comprises (2) contacting the first nucleic acid strand with a second primer that is complementary to the second primer binding site in the first nucleic acid strand under conditions in which a complementary strand of the first nucleic acid strand is synthesized. The amplification steps (1) and (2) are repeated, and the amplification produces a plurality of amplified molecules from the template strand, wherein the plurality of amplified molecules each have a different sequence at each end. As a result, a plurality of amplified second nucleic acid fragments is produced. The method further comprises characterizing the 5′ and 3′ end tags of the plurality of amplified second nucleic acid fragments.

As used herein, characterizing a nucleic acid sequence includes sequencing (partially or completely), karyotyping, polymorphism discovery or genotyping. Karyotyping is the analysis of the genome of a cell or organism. Polymorphism discovery or genotyping identifies differences between two or more nucleic acid sequences derived from different sources. In one embodiment, the nucleic acid sequence to be characterized is a genome. A genome is the genomic DNA of a cell or organism. In one embodiment, the genome is of a prokaryote, eukaryote, plant, virus, fungus, or an isolated cell thereof. In another embodiment, the genome is a known (previously characterized or sequenced) genome. In a further embodiment, the genome is an unknown (not previously characterized or sequenced) genome.

As used herein, fragmentation of a nucleic acid sequence or molecule can be achieved by any suitable method. These methods are generally referred to herein as the “fragmenting” of a nucleic acid sequence. For example, fragmenting of a nucleic acid sequence can be achieved by shearing (e.g. by mechanical means such as nebulization, hydrodynamic shearing through a small orifice, or sonication) the nucleic acid sequence or digesting the nucleic acid sequence with an enzyme, such as a restriction endonuclease or a non-specific endonuclease, or combinations thereof. In one embodiment, nucleic acid sequence fragments are produced by shearing of larger nucleic acid sequences (e.g., a genome) and the sheared fragments are subsequently treated (healed, or blunt-ended) to produce blunt ends. Any suitable method for blunt-ending of nucleic acid sequences can be used, e.g., treatment with one or more of the following: DNA polymerase in the presence of all four native 2′ deoxynucleoside 5′ triphosphates, DNA polymerase having a 3′ single-stranded exonuclease activity, a 3′ or 5′ single stranded DNA specific exonuclease, polynucleotide kinase, a single stranded DNA specific endonuclease, as will be understood by the person of skill in the art. The nucleic acid sequence fragments obtained can be of any size (e.g., molecular weight, length, etc.). In one embodiment, nucleic acid sequence fragments of a specific size (e.g., approximately greater than about 1 mb, about 200 kb, about 100 kb, about 80 kb, about 50 kb, about 20 kb, about 10 kb, about 3 kb, about 1.5 kb, about 1 kb, about 500 bases, about 200 bases and ranges thereof) are fractionated, for example, by gel electrophoresis or pulsed field gel electrophoresis, and isolated by any one of a variety of purification methods including, for example, electro-elution, enzymatic or chemical gel dissolution and extraction, mechanical gel disruption and extraction, dialysis, filtration, chromatography, or by other fractionation methods that are standard in the art.

As used herein, “joining” refers to methods such as ligation, annealing or recombination used to adhere one component to another. Recombination can be achieved by any methods known in the art. For example, recombination can be a Cre/Lox recombination. In one embodiment, the recombination is a between a pair of mutant lox sites that render the recombination unidirectional. In a further embodiment, the pair of mutant lox sites comprise a lox71 site and a lox66 site. In another embodiment, joining of a nucleic acid sequence to another nucleic acid sequence is performed by intermolecular ligation. For example, two nucleic acid sequences can be joined to form one contiguous nucleic acid sequence. A typical example of intermolecular ligation is cloning a nucleic acid sequence into a vector. A vector is generally understood in the art, and is understood to contain an origin of replication (“ori”) and a selectable marker for cloning DNA molecules in a bacterial host, such as Escherichia coli. In another embodiment, intermolecular ligation can be achieved using a non-vector nucleic acid. For example, an oligonucleotide such as a linker or an adapter can be intermolecularly ligated to the nucleic acid sequence of interest to facilitate isolation and amplification of that nucleic acid sequence.

As used herein, “without cloning” means that a nucleic acid sequence is isolated and/or amplified without the use of a vector and without any passage through a bacterial host cell. Isolation and amplification of nucleic acid sequences without cloning is advantageous because it avoids any interaction with the host cell DNA replication, recombination or expression machinery, which cause certain sequences to be lost from the cell, or propagated with low efficiency

In another aspect of the invention, provided herein is a method for producing a paired end library from a nucleic acid sequence using COS linkers and packaging into a bacteriophage. A “paired end library” is a plurality of paired ends from a plurality of fragments of a contiguous nucleic acid sequence. As used herein, a “paired end” (also referred to herein as a “paired tag”) is a nucleic acid sequence comprising a 5′ end of a contiguous nucleic acid sequence paired or joined with the 3′ end of the same nucleic acid sequence, wherein a portion of the internal sequence of the contiguous nucleic acid sequence is removed. COS linkers are linkers that comprise a COS site. In a particular embodiment, the COS site is a functional COS site, wherein the COS site is recognized by the enzymes present in a lambda DNA packaging extract and cleaved properly during packaging into a bacteriophage head. Packaging extracts are commercially available and known in the art (e.g., the Gigapack® lambda packaging extract available from Stratagene®).

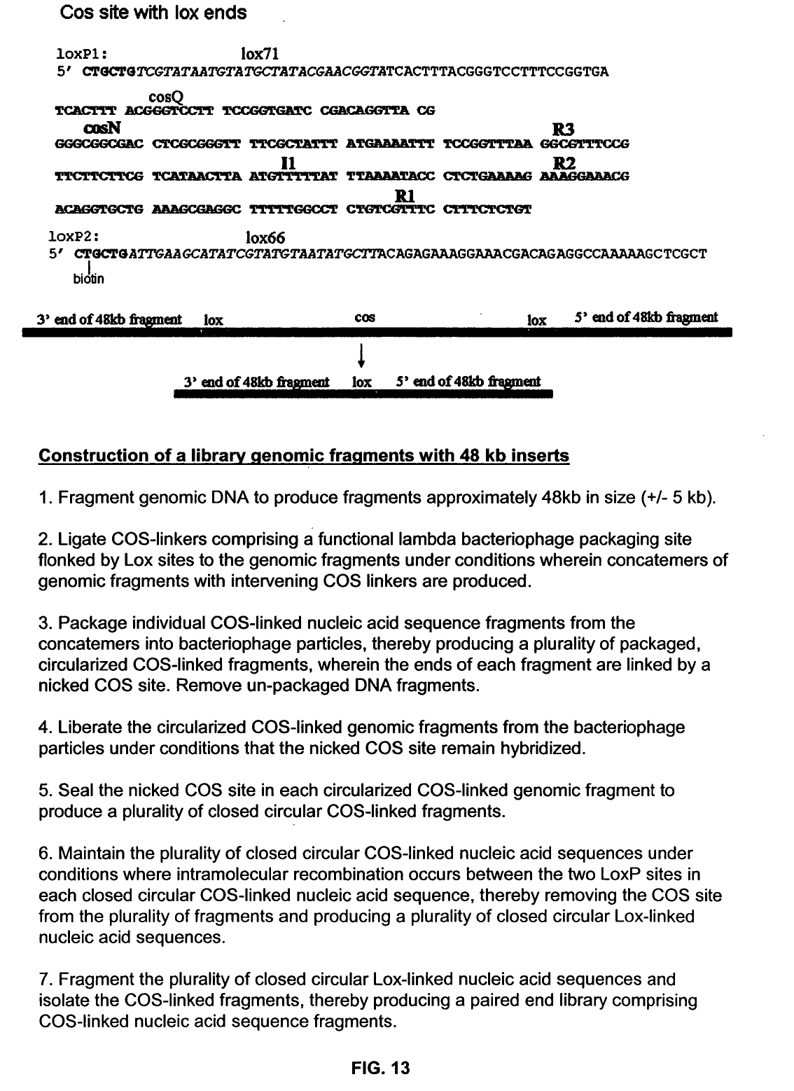

The method for producing a paired end library from a nucleic acid sequence using COS linkers and packaging into a bacteriophage comprises fragmenting a nucleic acid sequence to produce a plurality of nucleic acid sequence fragments of an appropriate size for packaging into a bacteriophage head, such as a lambdoid bacteriophage. COS-linkers comprising a functional COS site are ligated to the plurality of nucleic acid sequence fragments under conditions in which concatemers of nucleic acid sequence fragments and COS linkers are produced. The concatemers comprise the nucleic acid sequence fragments joined by COS linkers. Individual COS-linked nucleic acid sequence fragments from the concatemer are packaged into bacteriophage particles, wherein packaging results in cleavage and circularization of nucleic acid sequences that are flanked on both sides by COS sites that are in the same orientation, thereby producing a plurality of packaged, circularized COS-linked nucleic acid sequences, wherein the ends of each nucleic acid sequence fragment are linked by a nicked COS site. After packaging, unpackaged nucleic acid sequence fragments are destroyed, or alternatively, the bacteriophage particles containing packaged nucleic acid sequence fragments are isolated. The circularized COS-linked nucleic acid sequences within the bacteriophage particles are then liberated (e.g., released) from the particles by lysis under gentle conditions wherein the nicked COS sites remain hybridized (e.g., by treatment with proteinase K in 50 mM Tris-acetate, 50 mM sodium acetate, pH 7.5, at 37° C.). The nicked COS site in each circularized COS-linked nucleic acid sequence is then sealed with DNA ligase to produce a plurality of closed circular COS-linked nucleic acid sequences (e.g., by inactivating the proteinase K using phenyl methyl sulfonyl fluoride, and adding T4 DNA ligase with a sufficient amount of magnesium chloride and ATP to achieve a final concentration of 10 mM, each). The plurality of closed circular COS-linked nucleic acid sequences are then fragmented, thereby producing a paired end library from a nucleic acid sequence comprising COS-linked nucleic acid sequence fragments. A concatemer of nucleic acid sequence fragments and COS linkers is schematically shown in FIG. 13.

In one embodiment, the appropriate size of the nucleic acid sequence fragments for packaging into a lambdoid bacteriophage head, in conjunction with a COS-linker of about 200 bp, is about 48 kb +/−about 5 kb. In a preferred embodiment, the COS-linkers further comprise an affinity tag. An affinity tag is selected from the group consisting of biotin, digoxigenin, a hapten, a ligand, a peptide and a nucleic acid. In a further embodiment, COS-linked nucleic acid sequence fragments are isolated by capturing the affinity tag. In another embodiment of the invention, the COS-linker further comprises a selectable marker. As already noted, a selectable marker includes an antibiotic resistance gene, such as beta-lactamase, kanamycin resistance gene, ampicillin resistance gene, tetracycline resistance gene chloramphenicol.

In a particular embodiment, the plurality of closed circular COS-linked nucleic acid sequences are fragmented by shearing. In further embodiment, the plurality of closed circular COS-linked nucleic acid sequences are fragmented by shearing are subsequently blunt-ended (also referred to herein in “healed”). In another embodiment, the COS linker further comprises a restriction endonuclease recognition site for a restriction endonuclease that cleaves a nucleic acid sequence distally to the restriction endonuclease recognition site. Distally cleaving the nucleic acid sequence produces a 5′ end tag and/or a 3′ end tag. In one embodiment, the restriction endonuclease that cleaves a nucleic acid sequence distally to the restriction endonuclease recognition site is a TypeIIS or Type III restriction endonuclease. Thus, in one embodiment, the plurality of closed circular COS-linked nucleic acid sequences are fragmented by cleavage with a TypeIIS or Type III restriction endonuclease.

As used herein, “restriction endonucleases that cleave a nucleic acid distally to its restriction endonuclease recognition site” refers to a restriction endonuclease that recognizes a particular site within a nucleic acid sequence and cleaves this nucleic acid sequence outside the region of the recognition site (cleavage occurs at a site which is distal or outside the site recognized by the restriction endonuclease). In one embodiment, a restriction endonuclease that cleaves a nucleic acid distally to its restriction endonuclease recognition site cleaves on one side of the restriction endonuclease recognition site (for example, upstream or downstream of the recognition site). In another embodiment, restriction endonuclease that cleaves a nucleic acid distally to its restriction endonuclease recognition site cleaves on both sides of the restriction endonuclease recognition site (for example, upstream and downstream of the recognition site). In another embodiment, the restriction endonuclease cleaves once between two restriction endonuclease recognition sites. Examples of such restriction endonucleases are well known in the art, and include the following classes:

Type I (e.g., EcoKI, EcoAI, EcoBI, CfrAI, Eco377I, HindI, KpnA, IngoA V, StyLTII, StyLTIII, StySKI and StySPI) where the recognition sequence is bipartite and interrupted, and the cleavage site is distant and variable from recognition site, for example EcoKI:

(SEQ ID NO:8)

AAC (N6) GTGC (N > 400) /
TTG (N6) CACG (N > 400) /

where “/” designates the cut site,

Type IIs (e.g., AlwI, Alw26I, BbvI, BpmI, BsgI, BsrI, Earl, FokI, Hph I, MmeI, MboII SfaNI, Tth111I) where the recognition sequence is non-palindromic, nearly always contiguous and without ambiguities, and the cleavage site cuts in a defined manner with at least one cleavage site outside of the recognition sequence, for example:

Fok I:

(SEQ ID NO:9)

GGATG (N) 9 /

(SEQ ID NO:25)

CCTAC (N) 13 /

where “/” designates the cut site,

Type IIb (e.g. AlfI, AloI, BaeI, BcgI, BplI, BsaXI, BslFI, Bsp24I, CjeI, CjePI, CspCI, Fall, HaeIV, Hin4I, PpiI, and PsrI) where the recognition sequence is bipartite and interrupted, and the cleavage site cuts both strands on both sides of recognition site a defined, symmetric, short distance away and leaves 3′ overhangs; for example Bcg I:

(SEQ ID NO:10)

/10 (N) CGA (N) 6TCG (N) 12 /

(SEQ ID NO:11)

/12 (N) GCT (N) 6ACG (N) 10 /

where “/” designates the cut site,

Type III (e.g., EcoP I, EcoP15I, Hine I, Hinf III, and StyLT I) where the recognition Sequence is non-palindromic, and the cleavage site cuts approximately 25 bases away from the recognition sequence, for example EcoP15I:

(SEQ ID NO:12)

CAGCAG (N) 25-26/
GTCGTC (N) 25-26/

where “/” designates the cut site, and

Type IV (e.g., Eco57I, BseMII) where the recognition sequence is non-palindromic and the cleavage site cuts both DNA strands outside the target site, for example Eco57I:

(SEQ ID NO:13)

5′-CTGAAG (N) 16 /

(SEQ ID NO:14)

3′-GACTTC (N) 14 /

where “/” designates the cut site, and

In another embodiment, the method for producing a paired end library from a nucleic acid sequence further comprises amplification of the isolated COS-linked nucleic acid sequence fragments, thereby producing a library of amplified COS-linked nucleic acid sequence fragments. Thus, in one embodiment, the amplification comprises ligating a pair of asymmetrical adapters to the ends of each COS-linked nucleic acid sequence fragment, wherein the pair of asymmetrical adapters comprise:

a first asymmetrical oligonucleotide adapter selected from the group consisting of:

(i) an asymmetrical tail adapter comprising a first ligatable end, and a second end comprising a single-stranded 3′ overhang of at least about 8 nucleotides;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region,
   and a second asymmetrical oligonucleotide adapter selected from the group consisting of:
(i) an asymmetrical tail adapter comprising a first ligatable end, and a second end comprising a single-stranded 5′ overhang of at least about 8 nucleotides, wherein the 3′ end of the strand that does not comprise the 5′ overhang comprises at least one blocking group;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region.
   In the method, the first and second asymmetrical oligonucleotide adapters are not identical. When the pair of asymmetrical adapters are ligated to the ends of each COS-linked nucleic acid sequence fragment, an end-linked nucleic acid sequence fragment is produced. In one embodiment, the method further comprises amplifying one strand of the end-linked nucleic acid molecule referred to herein as the template strand. The amplification reaction comprises (1) contacting the template strand with a first primer that is complementary to a first primer binding site in a first asymmetrical adapter in the template strand. Under appropriate conditions, the first primer synthesizes a first nucleic acid strand in the amplification reaction, wherein the first nucleic acid strand is complementary to the template strand, and wherein the 3′ end of the first nucleic acid strand comprises a second primer binding site that is complementary to a sequence in the second asymmetrical adapter in the template strand. The amplification reaction further comprises (2) contacting the first nucleic acid strand with a second primer that is complementary to the second primer binding site in the first nucleic acid strand under conditions in which a complementary strand of the first nucleic acid strand is synthesized. The amplification steps (1) and (2) are repeated, and the amplification produces a plurality of amplified COS-linked nucleic acid fragment molecules from the template strand, wherein the plurality of amplified molecules each have a different sequence at each end. In one embodiment, the amplified COS-linked nucleic acid fragments are isolated by capturing the affinity tag. In a further embodiment, the plurality of amplified COS-linked nucleic acid fragments are sequenced.

In another aspect of the invention, provided herein is a method for producing a paired end library from a nucleic acid sequence. The method comprises fragmenting a nucleic acid sequence to produce a plurality of nucleic acid sequence fragments of an appropriate size for packaging into a lambdoid bacteriophage head. COS-linkers are ligated to the plurality of nucleic acid sequence fragments under conditions in which concatemers of nucleic acid sequence fragments and COS linkers are produced, wherein said COS-linkers comprise a functional COS site and two loxP sites flanking the functional COS site. Individual COS-linked nucleic acid sequence fragments from the concatemer are packaged into bacteriophage particles, thereby producing a plurality of packaged, circularized COS-linked nucleic acid sequences, wherein the ends of each nucleic acid sequence fragment are linked by a nicked COS site. The circularized COS-linked nucleic acid sequences are liberated from the bacteriophage particles under conditions that the nicked COS sites remain hybridized. The nicked COS site in each circularized COS-linked nucleic acid sequence are sealed to produce a plurality of closed circular COS-linked nucleic acid sequences. The plurality of closed circular COS-linked nucleic acid sequences are maintained under conditions suitable for intramolecular recombination between the two loxP sites in each closed circular COS-linked nucleic acid sequence, thereby removing the functional COS site from the plurality of closed circular COS-linked nucleic acid sequence fragments, thereby producing a plurality of closed circular lox-linked nucleic acid sequences. The plurality of closed circular lox-linked nucleic acid sequences are fragmented, thereby producing a paired end library from a nucleic acid sequence comprising lox-linked nucleic acid sequence fragments. In one embodiment, the appropriate size for packaging of the nucleic acid fragments into a lambdoid bacteriophage head is at least about 48 kb +/−about 4 kb. In another embodiment, the COS-linkers further comprise an affinity tag. An affinity tag can be selected from the group consisting of biotin, digoxigenin, a hapten, a ligand, a peptide and a nucleic acid. In one embodiment, the lox-linked nucleic acid sequence fragments are isolated by capturing the affinity tag. In another embodiment, the COS-linker further comprises a selectable marker. In a still further embodiment, the plurality of closed circular lox-linked nucleic acid sequences are fragmented by shearing. In one embodiment, the sheared plurality of closed circular lox-linked nucleic acid sequences are subsequently blunt-ended. In another embodiment, the COS-linker further comprises a restriction endonuclease recognition site for a restriction endonuclease that cleaves a nucleic acid sequence distally to the restriction endonuclease recognition site. The restriction endonuclease that cleaves a nucleic acid sequence distally to the restriction endonuclease recognition site can be, e.g., a Type I, TypeIIs, Type III or Type IV restriction endonuclease. Thus, in one embodiment, the plurality of closed circular lox-linked nucleic acid sequences are fragmented by cleavage with a Type I, TypeIIs, Type III or Type IV restriction endonuclease.

In a particular embodiment, the two loxP that flank a functional COS site in the COS-linker are mutated, such that recombination between the mutated sites renders one of the resulting recombined sites nonfunctional, thus making the recombination between the two loxP sites unidirectional. In one embodiment, the two mutated loxP sites are a lox71 site and a lox66 site (Oberdoerffer et al., 2003, Nucleic Acids Res. 15, e140).

In a further embodiment, the method for producing a paired end library from a nucleic acid sequence further comprises amplification of the isolated lox-linked nucleic acid sequence fragments, thereby producing a library of amplified lox-linked nucleic acid sequence fragments. Thus, in one embodiment, the amplification comprises ligating a pair of asymmetrical adapters to the ends of each lox-linked nucleic acid sequence fragment, wherein the pair of asymmetrical adapters comprise:

a first asymmetrical oligonucleotide adapter selected from the group consisting of:
(i) an asymmetrical tail adapter comprising a first ligatable end, and a second end comprising a single-stranded 3′ overhang of at least about 8 nucleotides;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region,
   and a second asymmetrical oligonucleotide adapter selected from the group consisting of:
(i) an asymmetrical tail adapter comprising a first ligatable end, and a second end comprising a single-stranded 5′ overhang of at least about 8 nucleotides, wherein the 3′ end of the strand that does not comprise the 5′ overhang comprises at least one blocking group;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region.
   In the method, the first and second asymmetrical oligonucleotide adapters are not identical. When the pair of asymmetrical adapters are ligated to the ends of each lox-linked nucleic acid sequence fragment, an end-linked nucleic acid sequence fragment is produced. The method further comprises amplifying one strand of the end-linked nucleic acid molecule referred to herein as the template strand. The amplification reaction comprises (1) contacting the template strand with a first primer that is complementary to a first primer binding site in a first asymmetrical adapter in the template strand. Under appropriate conditions, the first primer synthesizes a first nucleic acid strand in the amplification reaction, wherein the first nucleic acid strand is complementary to the template strand, and wherein the 3′ end of the first nucleic acid strand comprises a second primer binding site that is complementary to a sequence in the second asymmetrical adapter in the template strand. The amplification reaction further comprises (2) contacting the first nucleic acid strand with a second primer that is complementary to the second primer binding site in the first nucleic acid strand under conditions in which a complementary strand of the first nucleic acid strand is synthesized. The amplification steps (1) and (2) are repeated, and the amplification produces a plurality of amplified molecules from the template strand, wherein the plurality of amplified molecules each have a different sequence at each end. A plurality of amplified lox-linked nucleic acid fragments is thereby produced. In a further embodiment, the plurality of amplified lox-linked nucleic acid fragments are sequenced.

In the method of the invention, conditions that favor intramolecular ligation over intermolecular ligation are used when attempting to circularize DNA molecules in order to avoid chimeric ligation (i.e., the ligation of 5′ and 3′ ends from two different DNA molecules which results in the production of ditags). Conditions that favor intramolecular ligation over intermolecular ligation are known in the art. In one embodiment, intramolecular ligation is favored over intermolecular ligation by performing ligation at low DNA concentrations, and also in the presence of crowding reagents like polyethylene glycol (PEG) at low salt concentrations (Pfeiffer and Zimmerman, Nucl. Acids Res. (1983) 11(22): 7853-7871). Ligation at low DNA concentration can be expensive and impractical since large reaction volumes are used at high ligase concentration but dilute DNA concentration. The use of PEG increases the reaction rate, but long reaction times can still result in intermolecular products. In addition, volume exclusion does not eliminate diffusion of DNA molecules such that given enough time, DNA molecules will diffuse within reach of one another and ligate to one another. To overcome these problems, water-in-oil emulsions can be used. Water-in-oil emulsions have been described by Dressman et al. for single molecule PCR (Dressman et al., PNAS (2003), 100(15): 8817-8822). By creating a water-in-oil emulsion, billions of micro-reaction bubbles 10 micrometers in diameter, for example, can be generated. Using a dilute enough DNA concentration can ensure that only one or less than one molecule of DNA exists in any given micro-reactor. Under such conditions, long reaction times and additives (such as PEG, MgCl₂, DMSO) which increase the reaction rate of ligase (Alexander et al., Nuc. Acids Res. (2003) 31(12): 3208-3216) can be utilized without any risk of intermolecular ligation. Intramolecular ligation under such condition in an aqueous-in-oil emulsion is referred to herein as emulsion ligation.

In one embodiment, emulsion ligation of a nucleic acid sequence fragment is performed in the presence of a linker or adapter, such that the linker or adapter is incorporated into the resulting circular molecules between the 5′ and 3′ ends of the nucleic acid sequence fragment. In another embodiment, emulsion ligation of a nucleic acid sequence fragment is performed in the presence of a substrate, for example, a magnetic bead coupled to a linker or adaptor, such that the resulting circularized DNA becomes immobilized (covalently or non-covalently) onto the substrate. In each of these embodiments, the concentration of nucleic acid sequence fragments, linkers or adapters, and beads can be modulated independently to maximize intramolecular ligation or, if relevant, immobilization of an individual nucleic acid sequence fragment onto a single bead.

In another embodiment, emulsion ligation of a nucleic acid sequence fragment is performed in the presence of a substrate or a support, for example, a magnetic bead coupled to a linker or adaptor, such that the resulting circularized DNA becomes immobilized onto the substrate or support. In each of these embodiments, the concentration of nucleic acid sequence fragments, linkers or adapters, and beads can be modulated independently to maximize intramolecular ligation or, if relevant, immobilization of an individual nucleic acid sequence fragment onto a single bead. As used herein, “immobilized” means attached to a surface by covalent or non-covalent attachment means, as understood in the art. As used herein, a “substrate” is a solid or polymeric support such as a silicon or glass surface, a magnetic bead, a semisolid bead, a gel, or a polymeric coating applied to the another material, as is understood in the art.

Circularized nucleic acid molecules produced by intramolecular ligation with an intervening linker may be purified by a variety of methods known in the art, such as by gel electrophoresis, or by treatment with an exonuclease (e.g., Bal31 or “plasmid-safe” DNase) to remove contaminating linear molecules. Nucleic acid molecules incorporating a linker between the 5′ and 3′ ends of the starting nucleic acid sequence fragment can be purified by affinity capture using a number of methods known in the art, such as the use of a DNA binding protein that binds to the linker specifically, by triplex hybridization using a nucleic acid sequence complementary to the linker, or by means of a biotin moiety covalently attached to the linker (or adapter). Affinity capture methods typically involve the use of capture reagents attached to a substrate such as a solid surface, magnetic bead, or semisolid bead or resin.

In another aspect of the invention, provided herein is a cleavable adapter comprising an affinity tag and a cleavable linkage, wherein cleaving the cleavable linkage produces two complementary ends, and wherein the cleavable linkage is not a restriction endonuclease cleavage site. In one embodiment, the affinity tag is selected from the group consisting of biotin, digoxigenin, a hapten, a ligand, a peptide and a nucleic acid. In another embodiment, the cleavable adapter comprises a restriction endonuclease recognition site specific for a restriction endonuclease that cleaves a nucleic acid sequence distally to the restriction endonuclease recognition site. In another embodiment, the cleavable linkage in the cleavable adapter is a 3′ phosphorothiolate linkage. A 3′ phosphorothiolate linkage is illustrated by the general structure:

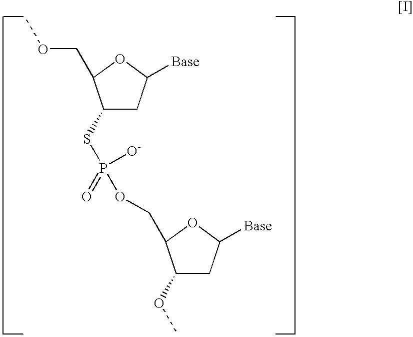

In another embodiment, the cleavable linkage in the cleavable adapter is a deoxyuridine nucleotide.

In another aspect of the invention, provided herein is a method for producing a paired tag library from a nucleic acid sequence. The method comprises fragmenting a nucleic acid sequence thereby producing a plurality of large nucleic acid sequence fragments of a specific size range. A cleavable adapter is introduced onto each end of each nucleic acid sequence fragment, wherein the cleavable adapter comprises an affinity tag and a cleavable linkage. The cleavable adapter attached to each end of each nucleic acid sequence fragment is cleaved, thereby producing a plurality of nucleic acid sequence fragments having compatible ends. The nucleic acid sequence fragments having compatible ends are maintained under conditions in which the compatible ends intramolecularly ligate, thereby producing a plurality of circularized nucleic acid sequences. The plurality of circularized nucleic acid sequences are fragmented, thereby producing a plurality of paired tags comprising a linked 5′ end tag and a 3′ end tag of each nucleic acid sequence fragment, which is a paired tag library produced from a plurality of large nucleic acid sequence fragments. In one embodiment, the specific size range of the large nucleic acid fragments is from about 2 to about 10 kilobase pairs, from about 10 to about 50 kilobase pairs, or from about 50 to 200 kilobase pairs, where a range of different size classes with a fairly tight distribution within each is useful to facilitate whole genome assembly (e.g., 3 kb +/−150 bp, 10 kb +/−500 bp, 48 kb +/−2 kb, 110 kb +/−5 kb). In a specific embodiment, the large nucleic acid sequence fragments are produced by shearing, blunt-ending, size fractionation and purification as understood in the art. In a further embodiment, the plurality of circularized nucleic acid sequences are sheared to produce the plurality of paired tags comprising a linked 5′ end tag and a 3′ end tag of each nucleic acid sequence fragment. In a still further embodiment, the plurality of paired tags comprising a linked 5′ end tag and a 3′ end tag of each nucleic acid sequence fragment are blunt-ended. In another embodiment, the cleavable adapter further comprises a restriction endonuclease recognition site specific for a restriction endonuclease that cleaves a nucleic acid sequence distally to the restriction endonuclease recognition site. Thus, in one embodiment, the plurality of circularized nucleic acids are cleaved by a restriction endonuclease that cleaves the nucleic acid sequence fragment distally to the restriction endonuclease recognition site.

In one embodiment, the cleavable adapter comprises an affinity tag selected from the group consisting of biotin, digoxigenin, a hapten, a ligand, a peptide and a nucleic acid. Thus, in one embodiment, the plurality of paired tags comprising the linked 5′ end tag and a 3′ end tag of each nucleic acid sequence fragment are isolated by capturing the affinity tags, thereby producing an isolated paired tag library. In another embodiment, the method for producing a paired tag library from a nucleic acid sequence further comprises amplification of the isolated paired tag library to produce a library of amplified paired tags. Thus, in one embodiment, amplification comprises ligating a pair of asymmetrical adapters to the ends of each paired tag, wherein the pair of asymmetrical adapters comprise:

a first asymmetrical oligonucleotide adapter selected from the group consisting of:

(i) an asymmetrical tail adapter comprising a first ligatable end, and a second end comprising a single-stranded 3′ overhang of at least about 8 nucleotides;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region,
   and a second asymmetrical oligonucleotide adapter selected from the group consisting of:
(i) an asymmetrical tail adapter comprising a first ligatable end, and a second end comprising a single-stranded 5′ overhang of at least about 8 nucleotides, wherein the 3′ end of the strand that does not comprise the 5′ overhang comprises at least one blocking group;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region.
   In the method, the first and second asymmetrical oligonucleotide adapters are not identical. When the pair of asymmetrical adapters are ligated to the ends of each paired tag, an end-linked nucleic acid sequence fragment (end-linked paired tag) is produced. Thus, the plurality of end-linked paired tags is a library of end-linked paired tags. The library of end-linked paired tags are amplified. Thus, the method further comprises amplifying one strand of the end-linked nucleic acid molecule referred to herein as the template strand. The amplification reaction comprises (1) contacting the template strand with a first primer that is complementary to a first primer binding site in a first asymmetrical adapter in the template strand. Under appropriate conditions, the first primer synthesizes a first nucleic acid strand in the amplification reaction, wherein the first nucleic acid strand is complementary to the template strand, and wherein the 3′ end of the first nucleic acid strand comprises a second primer binding site that is complementary to a sequence in the second asymmetrical adapter in the template strand. The amplification reaction further comprises (2) contacting the first nucleic acid strand with a second primer that is complementary to the second primer binding site in the first nucleic acid strand under conditions in which a complementary strand of the first nucleic acid strand is synthesized. The amplification steps (1) and (2) are repeated, and the amplification produces a plurality of amplified molecules from the template strand, wherein the plurality of amplified molecules each have a different sequence at each end. An amplified library of paired tags is thereby produced. In one embodiment, the amplified library of paired tags are sequenced. In another embodiment, the paired tag library is produced from a nucleic acid sequence that is a genome. In another embodiment, the cleavable linkage in the cleavable adapter is a 3′ phosphorothiolate linkage. Thus, in one embodiment, 3′ phosphorothiolate linkage is cleaved by Ag+, Hg2+ or Cu2+, at a pH of at least about 5 to at least about 9, and at a temperature of at least about 22° C. to at least about 37° C. In another embodiment, the cleavable linkage in the cleavable adapter is a deoxyuridine nucleotide. Thus, in one embodiment, the deoxyuridine is cleaved by uracil DNA glycosylase (UDG) and an AP-lyase.

In another aspect of the invention, provided herein are kits. The kits comprise one or more of the asymmetrical adapters as described herein. In particular embodiments, the kit comprises a pair of asymmetrical oligonucleotide adapters selected from the group consisting of:

a first asymmetrical oligonucleotide adapter selected from the group consisting of:

(i) an asymmetrical tail adapter comprising a first ligatable end, and a second end comprising a single-stranded 3′ overhang of at least about 8 nucleotides;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region,
   and a second asymmetrical oligonucleotide adapter selected from the group consisting of:
(i) an asymmetrical tail adapter comprising a first ligatable end, and a second end comprising a single-stranded 5′ overhang of at least about 8 nucleotides, wherein the 3′ end of the strand that does not comprise the 5′ overhang comprises at least one blocking group;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region.

In another embodiment, the kits further comprise a DNA ligase and buffer with required cofactors for the DNA ligase. In a further embodiment, the kits further comprise a first primer complementary to at least a portion of the single-stranded or unpaired region of said first asymmetrical oligonucleotide adapter, a second primer identical to at least a portion of the 5′ single-stranded or unpaired region of said second asymmetrical oligonucleotide adapter, a DNA polymerase suitable for performing PCR a mixture of 2′ deoxynucleoside 5′ triphosphates and a buffer with required cofactors for the DNA polymerase.

### EXAMPLE 1 ASYMMETRICAL ADAPTERS

In FIGS. 1A-C, the novel adapters of the present invention are schematically represented. FIG. 1A is a schematic representation of a 3′ asymmetrical tail adapter and 5′ asymmetrical tail adapter, each having a double-stranded region (5) ligated to a DNA fragment (insert) via a ligatable end (7). The 3′ asymmetrical tail adapter has a 3′ overhang (1), and the 5′ asymmetrical tail adapter has a 5′ overhang (2). FIG. 1B is a schematic representation of two different asymmetrical Y adapters, each having a double-stranded region (5) ligated to a DNA fragment (insert) via a ligatable end (7). Each asymmetrical Y adapter has two unpaired strands (1,2,3,4), each of which has a different sequence. FIG. 1C is a schematic representation of two different asymmetrical bubble adapters, each having a double-stranded region (5) ligated to a DNA fragment (insert) via a ligatable end (7). Each asymmetrical bubble adapter has an unpaired region wherein the unpaired strands (1,2,3,4) each have a different sequence. FIG. 1D is a schematic representation of 3 different types of ligatable ends of a double-stranded nucleic acid.

FIGS. 2A-C schematically illustrates the amplification of one strand of a nucleic acid sequence having a pair of asymmetrical tail adapters (A and B) ligated to the ends of a nucleic acid sequence using a primer (P1) which is complementary to unpaired (i.e., single-stranded) sequence (1) in tail adapter A (FIG. 1A) and a primer (P2) which is identical to unpaired sequence (2) in tail adapter B (FIG. 1A). The presence of a blocking group on asymmetrical tail adapter B (FIG. 2A) prevents extension of the tail adapter during amplification, thereby permitting amplification from only the primer P1.

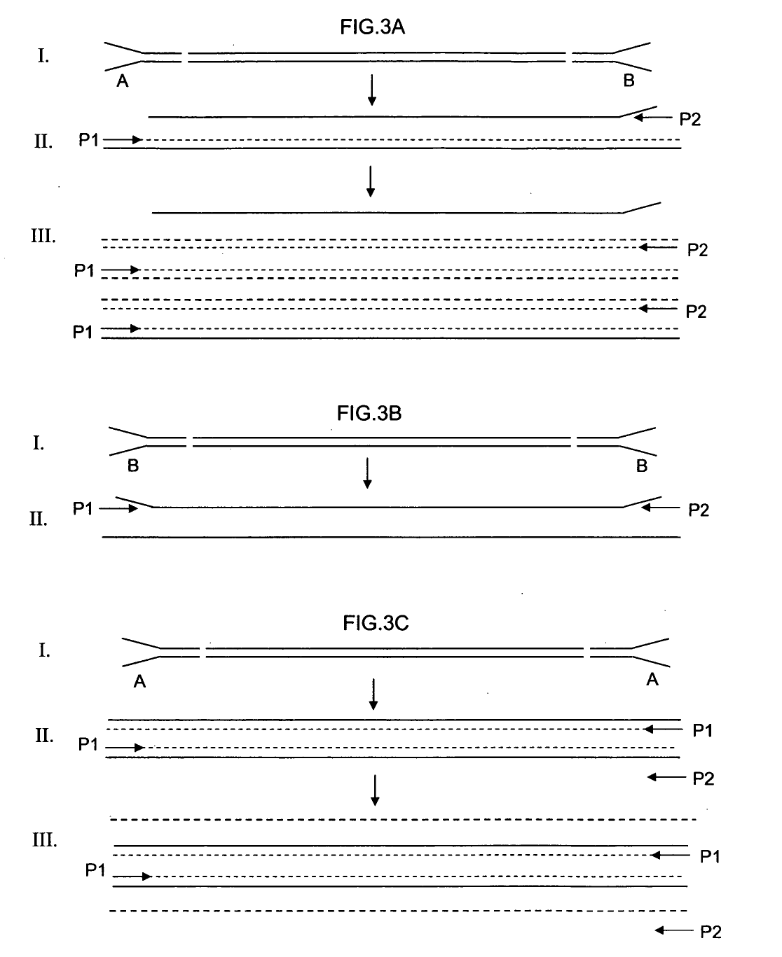

As illustrated in FIGS. 3A-C, similar results can be obtained by using a pair of Y-linkers together with a primer complementary to unpaired sequence (3) (FIG. 1B) and a primer identical to unpaired sequence (4) (FIG. 1B), or with a primer complementary to unpaired sequence (2) (FIG. 1B) and a primer identical to unpaired sequence (1) (FIG. 1B).

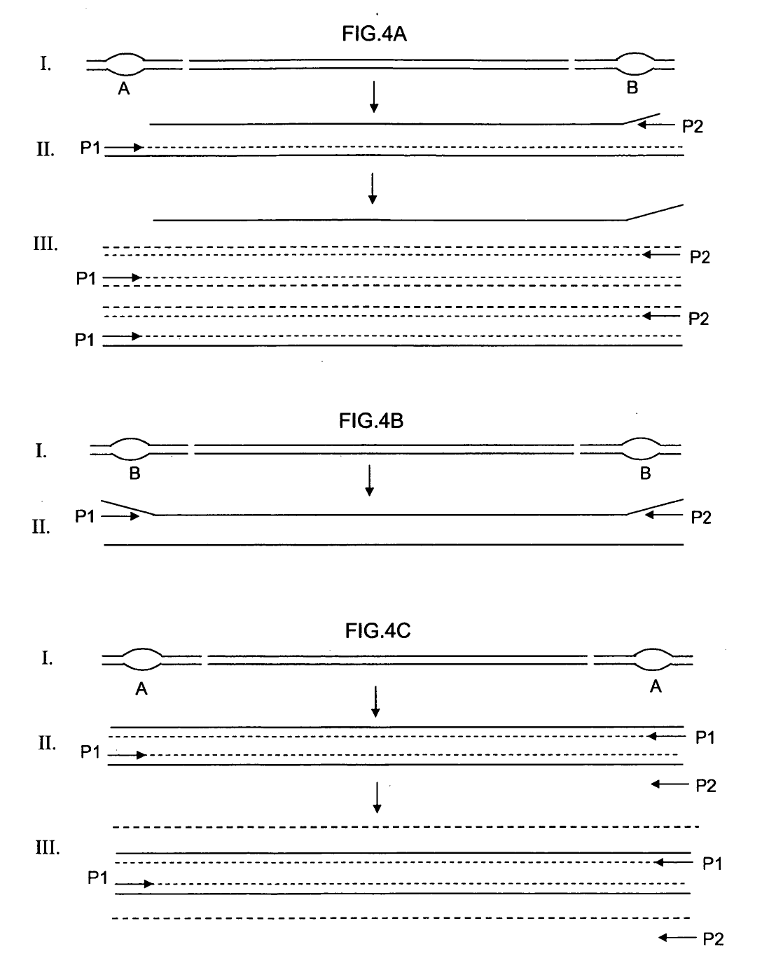

As illustrated in FIGS. 4A-C, similar results can also be obtained by using a pair of bubble-linkers together with a primer complementary to unpaired sequence (3) (FIG. 1C) and a primer identical to unpaired sequence (4) (FIG. 1C), or with a primer complementary to unpaired sequence (2) (FIG. 1C) and a primer identical to unpaired sequence (1) (FIG. 1C).

Similar results can also be obtained by using an appropriate mixture of tail linkers, Y-linkers and bubble-linkers with an appropriate selection of primers complementary to a 3′ unpaired sequence and identical to a 5′ unpaired sequence.

Another characteristic of these asymmetrical adapters is that they permit amplification of only one strand of the initial fragments that have adapters ligated to them. If the initial fragments have different structures or sequences at each end (e.g., a different 3′ overhang or 5′ overhang or blunt end resulting from a restriction endonuclease double-digest), then ligation of a pair of asymmetrical adapters having the complementary types of ligatable ends can be used to specifically enable amplification of only one strand of a given fragment with two different ends. The strand to be amplified (e.g., the tops strand or the bottom strand) can be selected by appropriate design of the tail adapters or by using alternate primer pairs for the Y- and bubble adapters (e.g., a pair consisting of a primer complementary to unpaired sequence (3) and a primer identical to unpaired sequence (4), or a pair consisting of a primer complementary to unpaired sequence (2) and a primer identical to unpaired sequence (1)).

### EXAMPLE 2 PCR CONFIRMATION OF SELECTIVE AMPLIFICATION

Several ligations and coupled ligation/PCR reactions were performed using asymmetric tail adapters selected from the following.

AsymA1:

(SEQ ID NO:15)

5′pCTGTCGTCTTGC
AsymA2:

(SEQ ID NO:16)

5′pGCAAGACGAGAGGTCCCACACGTAACACCAAACCTATCCACACTTTT
ACAAACCACTAGGACAGTCGCTACCTTAGTG
AsymA3:

(SEQ ID NO:17)

5′pGCAAGACGAGAGGTCCCACACGTAACACTAGGACAGTCGCTACCTTA
GTG
AsymA4:

(SEQ ID NO:18)

5′GTGTTACGTGTGGGACCTCTCGTCTTGG
AsymB1:

(SEQ ID NO:19)

5′-pCATCGTAC*T*C*T*ddCddCddC
AsymB2:

(SEQ ID NO:20)

5′CCTTAGGACCGTTATAGTTAGGTGCAGAAGCGAACACAGAGAGTAGGA
TG
AsymB3:

(SEQ ID NO:21)

5′CCTTAGGACCGTTATAGTTAGGTGGAGAGTAGGATG
AsymB4:

(SEQ ID NO:22)

5′pCATCCTACTCTCTGTGTTCG*C*T*T*ddCddCddC

Adapter A corresponds to a hybridization of AsymA2 and AsymA4 to form an asymmetrical tail adapter (adapter A); adapter A2 corresponds to a hybridization of AsymA3 and AsymA4 to form an asymmetrical tail adapter (adapter A2); and adapter B corresponds to a hybridization of AsymB1 and AsymB3 to form an asymmetrical tail adapter (adapter B). After hybridization to form the asymmetrical adapters, adapters A and B were ligated to each other and various amounts of the product were used as template for a PCR reaction conducted with 5 pmol each of primer complementary to the last 20 bp of AsymA2 and identical to the last 20 bp of AsymB2.

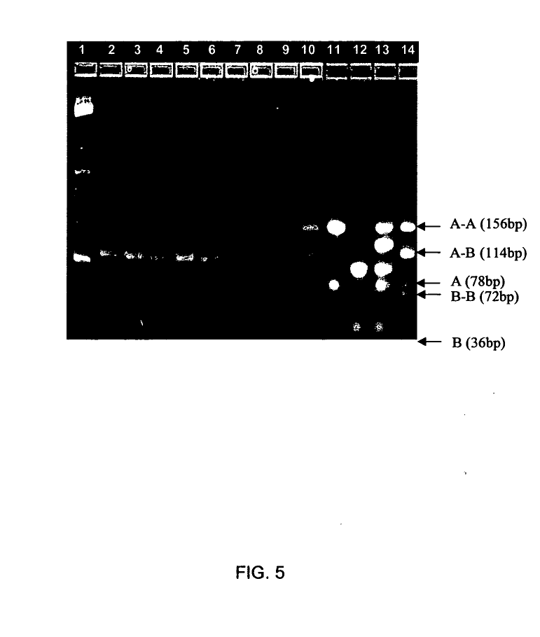

An aliquot of these ligation reactions were fractionated by electrophoresis on an agarose gel for size determination (see FIG. 5). A dilute amount of these ligation reactions were also amplified by PCR in accordance with the methods described herein. The results confirm that in the A-B ligation reaction, only a PCR product of the size A-B was obtained. The A-A and B-B products which are visible in the A-B ligation, are suppressed in the PCR and are not exponentially amplified, as described in Example 1.

### EXAMPLE 3 CONSTRUCTION OF A PAIRED END LIBRARY FROM E. COLI STRAIN DH10B USING MmeI OR EcoP15I ADAPTERS

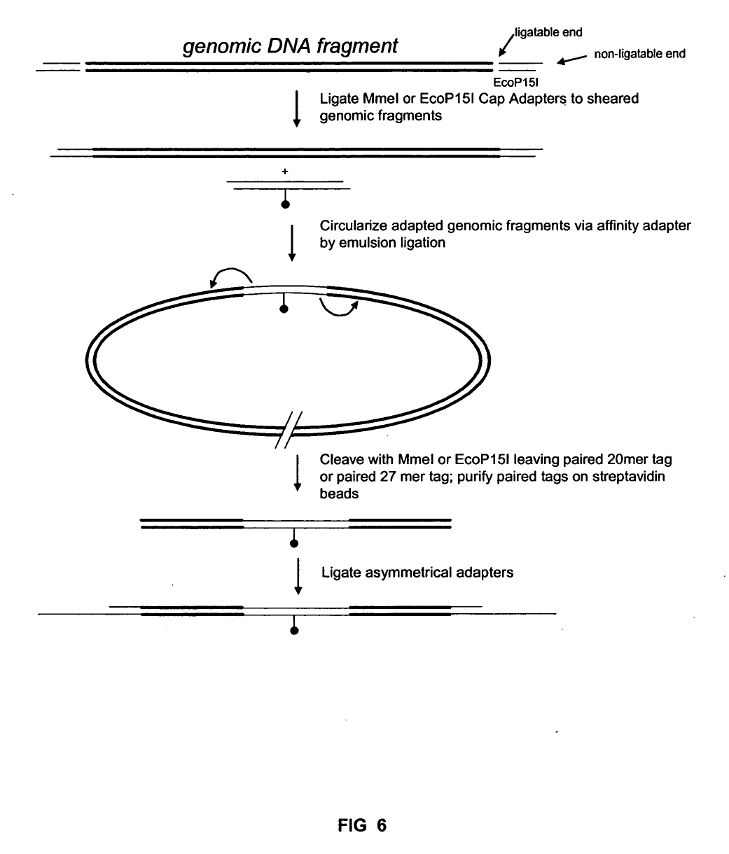

This example utilizes the strategy shown schematically in FIG. 6 to construct a representative library of amplified genomic DNA fragments with asymmetric adapters derived form the E. coli DH10B genome.

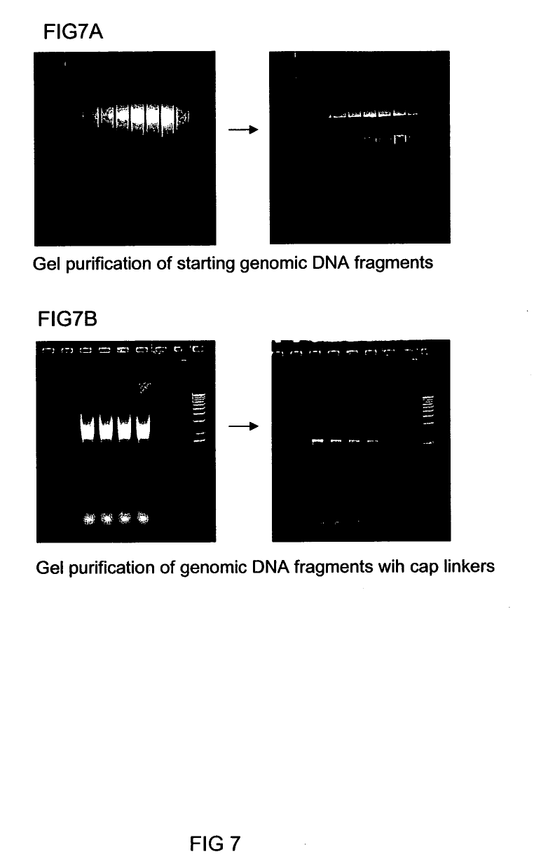

Ten miocrograms of genomic DNA from E. coli strain DH10b was randomly sheared on a Hydroshear machine, in a volume of 120 ul using shear Code 12 for 20 cyles. 60 ug of the sheared DNA was fractionated on a 1.2% TAE-Agarose gel and DNA fragments in a 1.8-4 kb size range were collected (Results shown in FIG. 7A).

The DNA fragments were extracted from gel using a Qbiogene GeneClean kit. 13.6 ug of sheared, sized selected DNA was recovered. The fragments were blunt-ended using a mixture of T4 DNA Polymerase, T4 Polynucleotide Kinase, dATP, dCTP, dGTP. dTTP and ATP (Epicentre ‘Endit’ Kit) under the following conditions:

136 ul sheared, sized selected DNA

20 ul Endit 10× buffer

20 ul Endit dNTPs

20 ul Endit ATP

4 ul Endit Enzyme mix

After incubation at room temperature for 40 min, the enzymes were inactivated by heating at 70C for 20 min followed by Phenol-Chloroform extraction, and the DNA was precipitated with ethanol.

The blunt-ended fragments were ligated (overnight at 16C) to asymmetrical tail adapters (referred to as “cap adapters” in FIG. 6). The tail adapters comprise one ligatable blunt end, an adjacent EcoP15I or MmeI restriction endonuclease recognition site, and a non-self-complemenatry overhang at the other end. The overhangs are complementary to the overhangs of a third adapter that comprises an affinity tag.

MmeI adapter Ligation:

95 ul DNA (9.5 ug)

25 ul 5× Invitrogen Ligase Buffer

3.5 ul MmeI Cap Adapter 500 uM

6 ul Invitrogen Ligase (1 u/ul)

EcoP15I adapter ligation:

35 ul DNA (3.5 ug)

10 ul % X Invitrogen Ligase buffer

1.3 ul EcoP15I Cap Adapter 500 uM

3 ul Invitrogen Ligase (1 u/ul)

The ligated fragments were fractionated on 1.2% agarose gel and the 1.8-4 kb fragments were excised to remove excess adapters (FIG. 7B).

The fragments were recovered from the agarose using a Geneclean kit, resulting ˜3.3 ug DNA from MmeI library and 2.5 ug from EcoP15I library

The adapter ligated fragments were ligated to an affinity linker at ˜1.3 ng/ul final DNA concentration and 3:1 affinity linker to insert ratio in order to achieve a high efficiency of intramolecular ligation (i.e., circularization).

Three MmeI ligations of:

34 ul DNA

60 ul 10× Epicentre Ligase Buffer

24 ul 25 mM Epicentre ATP

1.65 ul 1 pmol/ul Internal Affinity Adapter

476 ul dH20

6 ul Invitrogen Ligase (1 U/ul)

Three EcoP15I ligations of:

41 ul DNA

60 ul 10× Epicentre Ligase Buffer

24 ul 25 mM Epicentre ATP

0.25 ul 10 pmol/ul Internal Affinity Adapter

469 ul dH20

6 ul Invitrogen Ligase (1 U/ul)

The samples were incubated at 16C for 4 hr and the ligase was inactivated by incubation 65C for 15 min. The samples were then treated with PlasmidSafe exonuclease to remove all remaining linear DNA fragments by adding to each ligation:

5 ul 25 mM Epicentre ATP

5 ul PlasmidSafe Exonuclease (Epicentre)

The samples were incubated at 37C for 45 min. The exonuclease was inactivated by heating at 70C for 20 min, extracted with phenol-chloroform and precipitated with ethanol. The fragments were then digested with EcoP15I or MmeI at 37C for 1 hr as follows.

EcoP15I Digest:

120 ul DNA

20 ul NEB3 10×

20 ul NEB 10×ATP

20 ul 10× Sinefungin

2 ul 100×BSA

10 ul EcoP15I (2 U/ul)

6 ul dH20

MmeI Digest:

120 ul DNA

20 ul NEB4 10×

20 ul 10×SAM

35 ul dH20

5 ul MmeI

The enzymes were inactivated by incubation at 65C for 30 min, extracted with phenol-chloroform and precipitated with ethanol.

The fragments produced by EcoP15I digestion were treated to produce blunt ends by filling in with T4 polymerase in the Epicentre Endit kit.

34 ul DNA

5 ul 10 Expicentre Endit Buffer

5 ul Endit dNTPs

5 ul Endit ATP

1 ul Endit Enzyme Mix

The sample was incubated at room temperature for 40 min, heat killed 20 min at 70C, phenol-chloroform extracted and ethanol precipitated.

The blunt-ended fragments were then ligated to asymmetric adapters having a blunt ligatable end for EcoP15I library, or a 2 bp 3′ NN overhang for MmeI library. The ligation reactions contain:

75 ul DNA

20 ul 5× ligase buffer

5 ul ligase

0.5 ul 125 pmol/ul AsymA2,A4 (blunt) or AsymA1,A3 (2 bp 3′ overhang) insert:linker ratio ˜1:100

AsymA1:

(SEQ ID NO:15)

5′pCTCTCGTCTTGC
AsymA2:

(SEQ ID NO:16)

5′pGCAAGACGAGAGGTCCCACACGTAACACCAAACCTATCCACACTTTT
ACAAACCACTAGGACAGTCGCTACCTTAGTG
AsymA3:

(SEQ ID NO:17)

5′pGCAAGACGAGAGGTCCCACACGTAACACTAGGACAGTCGCTACCTTA
GTG
AsymA4:

(SEQ ID NO:18)

5′GTGTTACGTGTGGGACCTCTCGTCTTGC

AsymA4: 5′GTGTTACGTGTGGGACCTCTCGTCTTGC (SEQ ID NO: 18)

0.5 ul 125 pmol/ul AsymB1,B2 (blunt) or AsymB3,B4 (2 bp 3′ overhang)

AsymB1:

(SEQ ID NO:19)

5′pCATCCTAC*T*C*T*ddCddCddC
AsymB2:

(SEQ ID NO:20)

5′CCTTAGGACCGTTATAGTTAGGTGCAGAAGCGAACACAGAGAGTAGGA
TG
AsymB3:

(SEQ ID NO:21)

5′CCTTAGGACCGTTATAGTTAGGTGGAGAGTAGGATG
AsymB4:

(SEQ ID NO:22)

5′pCATCCTACTCTCTGTGTTCG*C*T*T*ddCddCddC

(Note: the ‘*’ symbol indicates a phosphorothioate linkage; ddC indicates a 2′3′-dideoxy-cytidine residue)

The samples were ligated at room temperature for 4 hrs, heat killed, phenol-chloroform extracted, ethanol precipitated and resuspend in 200 ul TE

The fragments containing an affinity adapter were then bound to streptavidin coated magnetic beads and contaminating fragments washed away:

To each extract add 200 ul 2× B&W

Wash 10 ul Dynal Streptavidin M280 beads with B&W

Remove solution from beads and add extracted library in I X B&W to beads

Rotate at Room Temperature 1 hr to bind

Wash 1× B&W 180 ul

Wash 1× Wash1E

3× Wash1E with 0.1% Tween 20 at 50C

transfer to fresh tube

wash 3× W1Etween20

wash 1× Low TE

2× dH20

The purified fragments were eluted in 18 ul dH20 by heating to 95C for 5 min followed by recovery of the eluate and repeatin the elution with a second 18 ul. The recovered fragments were amplified by PCR in areaction containing:

50 ul Invitrogen Platinum PCR Supermix

1 ul P1 Primer 50 uM 1 ul P2 Primer 50 uM

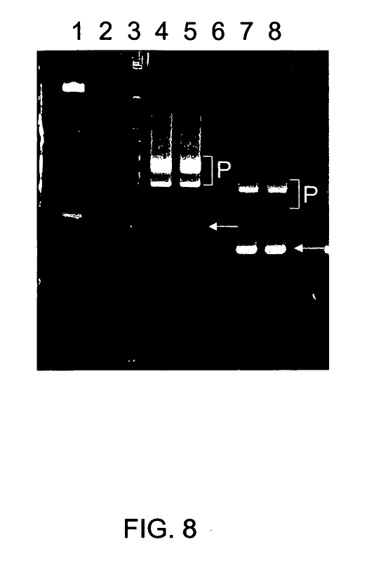

After thermal cycling for 32 cycles of PCR using the program: 95C 4 min, (95C 15 s, 55C 10 s, 70C 1 min)×32, 4C hold, the samples were evaluated on a 4% Invitrogen Egel. The results are shown in FIG. 8.

Products from each library were excised from the gel, purified using a GeneClean kit, cloned using an Invitrogen TOPO-TA cloning kit, and 200 clones were sequenced using the M13F primer using standard methods with detection on an ABI3730 xl automated sequencer. The sequencing verified the correct structure:
AsymA-Tag1-Affinity adapter-Tag2-AsymB

### EXAMPLE 4 PAIRED END LIBRARY CONSTRUCTION USING CLEAVABLE ADAPTERS

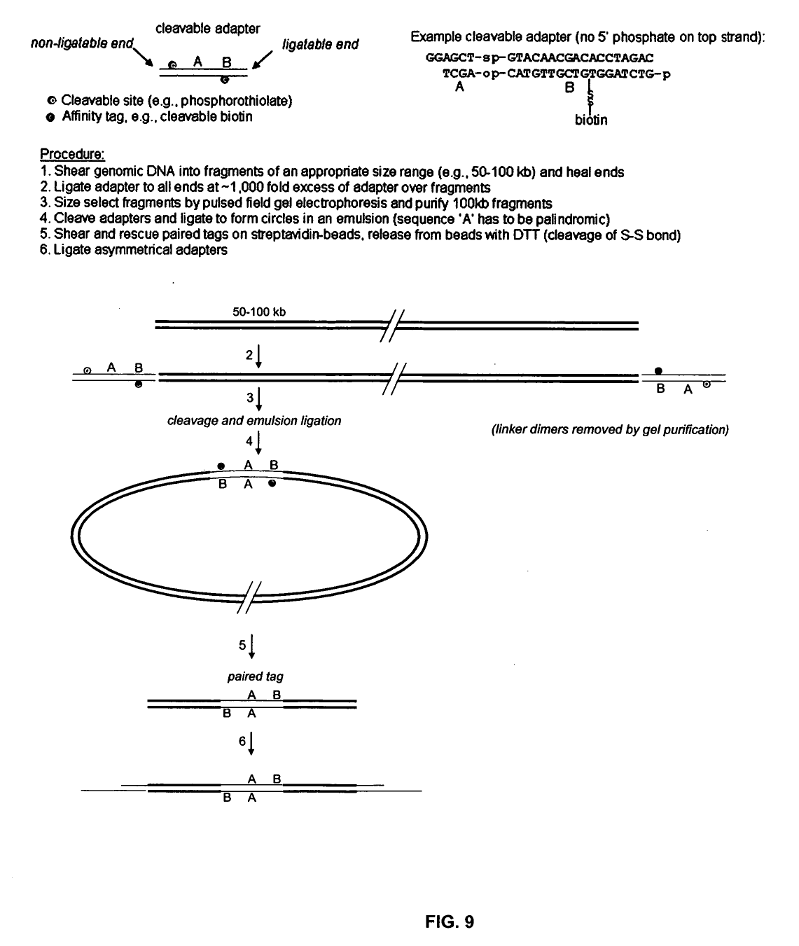

As shown in FIG. 9, a linker/adapter containing a chemically cleavable linkage and an affinity tag is used to modify the ends of the genomic DNA fragments initially produced by shearing of genomic DNA (those fragments are derived by shearing genomic DNA to a specific size range, e.g., about 50-100 kb and blunt-ending the fragments. The adapter contains a 5′ phosphate at one end, however, there is no 5′ phosphate at the other end. Optionally, the adapter contains some extra bases to further prevent any ligation from occurring at the end lacking the 5′ phosphate. After ligating the adapter onto the fragments, amplification will yield only the products of fragments with an adapter attached at each end or adapter dimers formed by ligation of two adapters together. DNA fragments of a defined size range with adapted ends are purified by after fractionation by pulsed field gel electrophoresis. This purification step also serves to remove the unwanted adapter dimers. The cleavable linkage is then cleaved (in the specific case shown, using silver nitrate to cleave a 3′ phosphorothiolate linkage) leaving a 5′ phosphate at each end of the linkerized fragments and a self-complementary 3′ overhang (this overhang could be any self-complementary sequence). The resulting fragments are then diluted to an appropriate concentration and circularized by intramolecular ligation in an aqueous-in-oil emulsion. The circularized molecules are recovered from the emulsion (e.g., by detergent or solvent addition) and are sheared to a smaller size (e.g., 500-1,000 bp). The fragments containing the paired tags are then recovered via affinity capture of the biotin tag on binding to streptavidin-coated magnetic beads and the excess fragments are washed away to produce a purified population of fragments containing paired tags. The use of a cleavable biotin moiety facilitates release of the fragments from the solid support (e.g. streptavidin-coated magnetic beads). Finally, the paired tag fragments are blunt-ended and asymmetrical adapters are ligated to enable amplification of a set of paired tags having a different adapter sequence at each end.

### EXAMPLE 5 METHOD FOR MAKING A ˜48 KB PAIRED TAG LIBRARY

The method allows construction of high quality paired end libraries from the ends of DNA fragments approximately 43-53 kb in length. It takes advantage of the Lambda phage packaging system to provide precise length control of the packaged DNA fragments, similar to that displayed by other lambda based cloning systems (e.g., cosmids and fosmids). The advantages are that no cloning vector is used and the cloned molecules are never passed through E. coli, so there is no cloning bias.

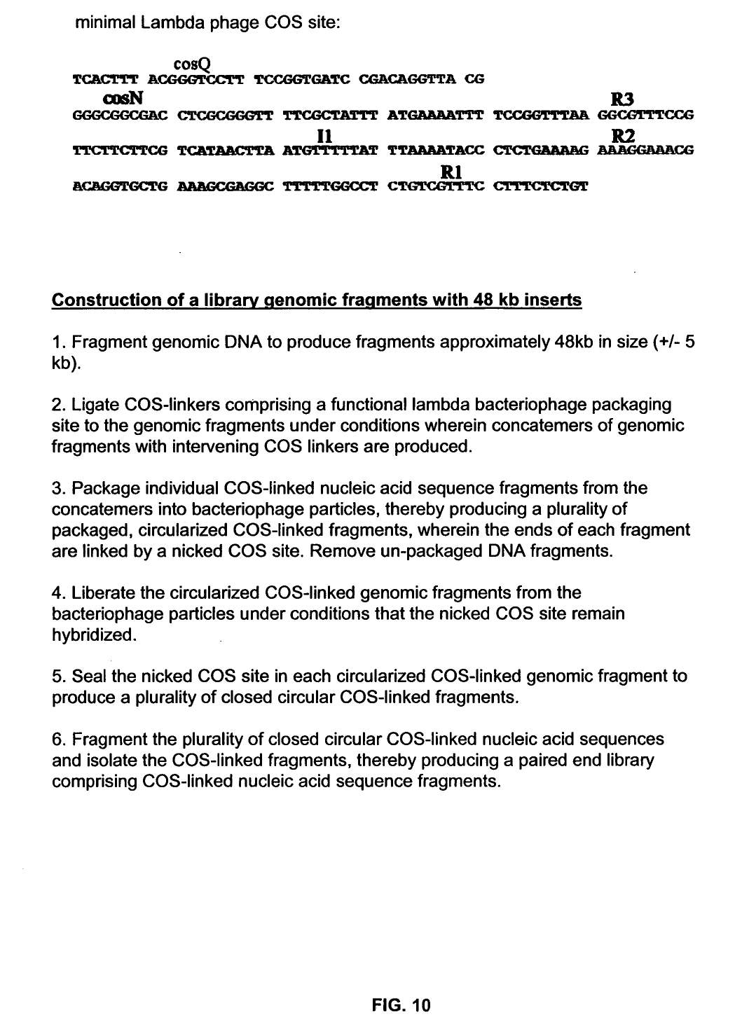

The overall procedure is outlined in FIG. 10. The method involves the following steps:

1. Fragment genomic DNA to produce fragments approximately 48 kb in size (+/−5 kb).

2. Ligate COS-linkers comprising a functional lambda bacteriophage packaging site to the genomic fragments under conditions wherein concatemers of genomic fragments with intervening COS linkers are produced.

3. Package individual COS-linked nucleic acid sequence fragments from the concatemers into bacteriophage particles, thereby producing a plurality of packaged, circularized COS-linked fragments, wherein the ends of each fragment are linked by a nicked COS site. Remove un-packaged DNA fragments.

4. Liberate the circularized COS-linked genomic fragments from the bacteriophage particles under conditions that the nicked COS site remain hybridized.

5. Seal the nicked COS site in each circularized COS-linked genomic fragment to produce a plurality of closed circular COS-linked fragments.

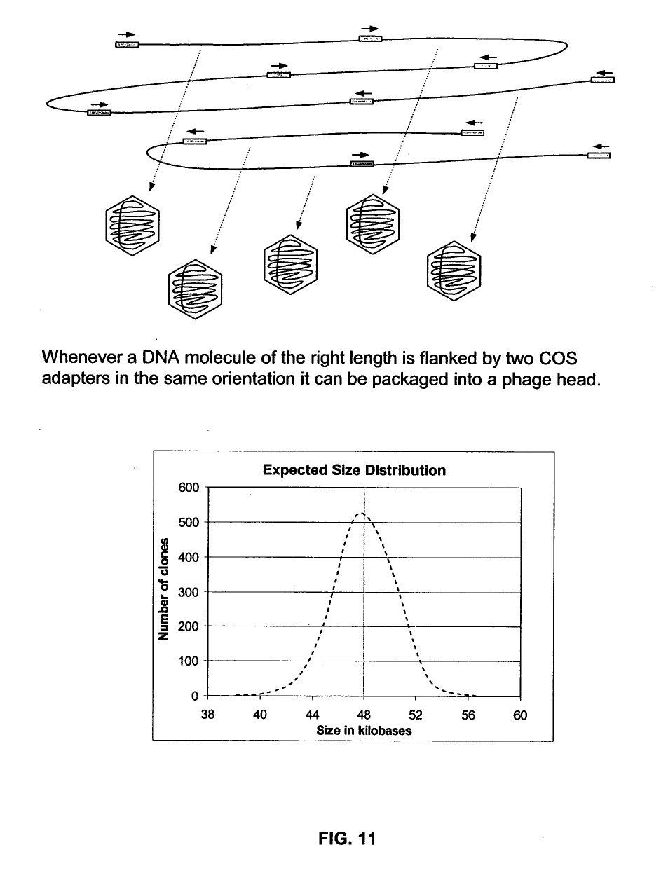

6. Fragment the plurality of closed circular COS-linked nucleic acid sequences and isolate the COS-linked fragments, thereby producing a paired end library comprising COS-linked nucleic acid sequence fragments. This method takes advantage of the affinity adapter and asymmetrical adapter (tail-adapter) approaches described herein. A schematic of the packaging substrate is illustrated in FIG. 11. When a DNA molecule of the correct size is flanked by two COS linkers (adapters) in the same orientation in the packaging substrate, the DNA molecule can be packaged into a phage head. The length of a functional COS site is approximately 200 bp.

The resulting paired tags, including the ends of the starting fragments with an intervening affinity adapter, is amplified by emulsion PCR or some other single molecule based method for use in a massively parallel sequencing approach (e.g., polony sequencing, 454 pyrosequencing, or Solexa colony sequencing). Alternatively, the paired tags can be cloned for analysis with conventional sequencing technology.

The complete sequence of a COS-linker is provided in FIG. 10, although, some sequence variation can be tolerated, as will be recognized by a person of skill in the art. A typical size distribution expected for a library packaged using lambda packaging extracts is illustrated in FIG. 11. This is based on a similar distribution for 40 kb fosmid clones produced by conventional fosmid cloning methods. By using a 200 bp COS fragment instead of an 8 kb fosmid vector the average insert size is expected to be 8 kb larger (or 48 kb, on average). Thus, this method provides a library that has a narrow and accurate size distribution.

### EXAMPLE 6 COS-LINKERS COMPRISING AN EcoP15I RECOGNITION SITE

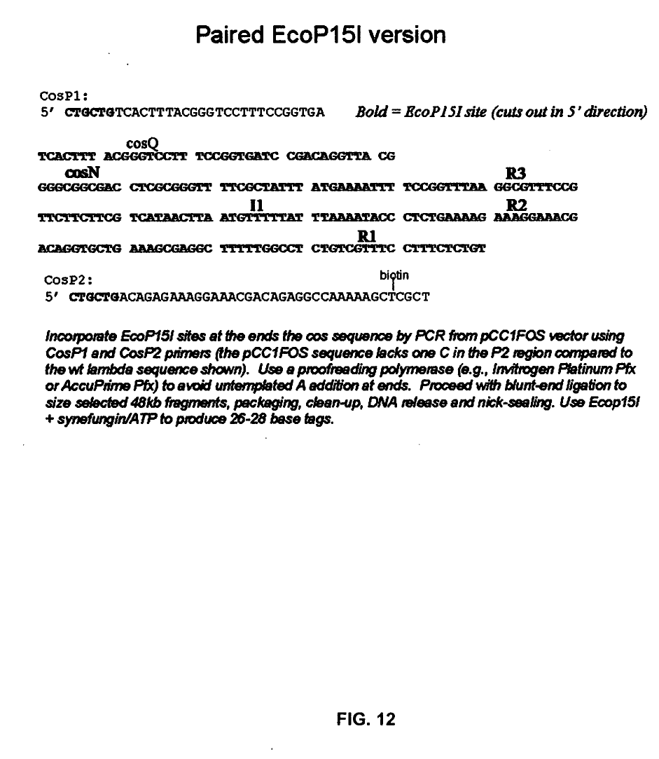

EcoP15I (or another type III or type IIS enzyme, such as MmeI) can be used to produce a short paired tag, as described herein. FIG. 12 illustrates how to create a Cos fragment with EcoP15I sites at the ends for ligation to genomic DNA prior to packaging.

### EXAMPLE 7 COS-LINKERS COMPRISING LOX P ENDS

LoxP sites permit excision of the Cos fragment after creation of the paired ends in the methods disclosed herein. This approach reduces the size of the final paired tag fragment which further facilitates emulsion PCR (long fragments are more difficult to amplify by emulsion PCR). In addition, retrieving a fragment with a shorter intervening sequence by affinity capture, permits the retention of a longer flanking genomic sequence tag on either side of the affinity tag (which in this case is the final loxP site). FIG. 13 illustrates how to create a Cos fragment with loxP ends. As described herein, the method for construction of a library of genomic fragments with approximately 48 kb inserts comprises the steps:

1. Fragment genomic DNA to produce fragments approximately 48 kb in size (+/−5 kb).

2. Ligate COS-linkers comprising a functional lambda bacteriophage packaging site flonked by Lox sites to the genomic fragments under conditions wherein concatemers of genomic fragments with intervening COS linkers are produced.

3. Package individual COS-linked nucleic acid sequence fragments from the concatemers into bacteriophage particles, thereby producing a plurality of packaged, circularized COS-linked fragments, wherein the ends of each fragment are linked by a nicked COS site. Remove un-packaged DNA fragments.

4. Liberate the circularized COS-linked genomic fragments from the bacteriophage particles under conditions that the nicked COS site remain hybridized.

5. Seal the nicked COS site in each circularized COS-linked genomic fragment to produce a plurality of closed circular COS-linked fragments.

6. Maintain the plurality of closed circular COS-linked nucleic acid sequences under conditions where intramolecular recombination occurs between the two LoxP sites in each closed circular COS-linked nucleic acid sequence, thereby removing the COS site from the plurality of fragments and producing a plurality of closed circular Lox-linked nucleic acid sequences.

7. Fragment the plurality of closed circular Lox-linked nucleic acid sequences and isolate the COS-linked fragments, thereby producing a paired end library comprising COS-linked nucleic acid sequence fragments.

### EXAMPLE 8 BAC END TAGS

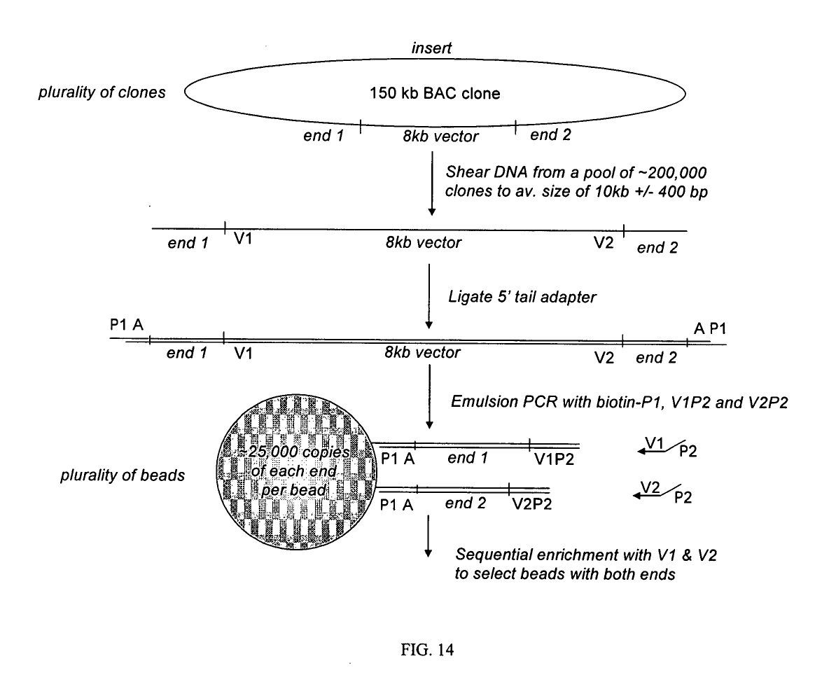

An asymmetrical linker of the present invention can also be used to characterize BAC end tags (or paired tags) produced as exemplified in FIG. 14. In this example, the asymmetrical linkers attached to each end of the paired end from the BAC insert can be identical and can be both tail adapters, Y adapters or bubble adapters. A tag is generated from a clone library, such as a BAC library (e.g., a commercially available BAC library). The BAC clones are fragmented (e.g., by shearing) to produce fragments of a size approximately 100 bp to about 2.5 kb larger than the BAC vector size. Preferably, the fragments are approximately 10 kb +/−about 400 bp when the vector size is 8 kb, wherein a number of the fragments will comprise the vector and a fragment of the insert nucleic acid sequence from the BAC clone at either end of the vector nucleic acid sequence (see FIG. 14). V1 and V2 represent the vector ends; end 1 and end 2 represent the fragments of the insert DNA ends attached to the vector. Asymmetrical adapters are ligated to the ends of the fragmented BAC clones (see FIG. 14; asymmetrical tail adapters (“AP1” and “1PA”, wherein 1PA represents the AP1 adapter in reverse orientation) are shown for illustration purposes, as indicated above, the adapter can be a tail adapter, a Y adapter or a bubble adapter). Amplification is performed using a primer (P1) which complementary to at least a portion of the single-stranded sequence in the adapter and two primers that are sequence specific for the two ends of the vector sequence (see FIG. 14, vector primers referred to as V1P2 and V2P2). Preferably, the vector primers are specific for a universal nucleic acid sequence in a vector (e.g., an SP6 and T7 sequences, as will be understood by a person of skill in the art). Furthermore, the P1 primer can comprise an affinity tag (e.g., biotin) which can be attached to a bead via avidin or streptavidin binding, for example, or the P1 primer can be attached directly to a bead. Further amplification can be performed to sequentially enrich for beads that contain nucleic acid sequences that comprise both vector ends using the vector-specific primers. The ends of the BAC library can be further characterized, such as sequenced.

While this invention has been particularly shown and described with references to preferred embodiments thereof, it will be understood by those skilled in the art that various changes in form and details may be made therein without departing from the scope of the invention encompassed by the appended claims.

1. A pair of asymmetrical oligonucleotide tail adapters comprising:
a) a first oligonucleotide tail adapter comprising a 3′ overhang; and
b) a second oligonucleotide tail adapter comprising a 5′ overhang with at least one blocking group at the 3′ end of the strand that does not comprise the 5′ overhang.

2. The first oligonucleotide tail adapter of claim 1, wherein the 3′ overhang comprises at least one primer binding site.

3. A pair of asymmetrical oligonucleotide tail adapters, comprising:
a) a first partially double-stranded oligonucleotide tail adapter comprising a ligatable end, and a 3′ single-stranded overhang of at least about 8 nucleotides at the opposite end; and
b) a second double-stranded oligonucleotide tail adapter comprising a ligatable end, and a 5′ single-stranded overhang comprising at least about 8 nucleotides at the opposite end, wherein the 3′ end of the strand that does not comprise the 5′ overhang comprises at least one blocking group.

4. The first partially double-stranded oligonucleotide tail adapter of claim 3,
wherein the single-stranded 3′ overhang comprises at least one primer binding site.

5. A pair of Y oligonucleotide adapters, comprising:
a) a first partially double-stranded Y oligonucleotide adapter comprising a first ligatable end, and a second unpaired end comprising two non-complementary strands, wherein the length of the non-complementary strands are at least about 8 nucleotides; and
b) a second partially double-stranded Y oligonucleotide adapter comprising a first ligatable end, and a second unpaired end comprising two non-complementary strands, wherein the length of the non-complementary strands are at least about 8 nucleotides,
wherein the nucleic acid sequence of the first and second double-stranded Y oligonucleotide adapters are not identical.

6. The pair of Y oligonucleotide adapters of claim 5, wherein at least one non-complementary strand of at least one Y oligonucleotide adapter comprises at least one primer binding site.

7. A pair of asymmetrical bubble oligonucleotide adapters, comprising:
a) a first partially double-stranded bubble oligonucleotide adapter comprising an unpaired region of at least about 8 nucleotides flanked on each side by a paired region; and
b) a second partially double-stranded bubble oligonucleotide adapter comprising an unpaired region of at least about 8 nucleotides flanked on each side by a paired region,
wherein the nucleic acid sequence of the first and second asymmetrical bubble oligonucleotide adapters are not identical.

8. The first double-stranded bubble oligonucleotide adapter of claim 7, wherein
the unpaired region comprises at least one primer binding site.

9. A pair of asymmetrical oligonucleotide adapters comprising:
a) a first oligonucleotide adapter selected from the group consisting of:
(i) an asymmetrical tail adapter comprising a first ligatable end, and a second end comprising a single-stranded 3′ overhang of at least about 8 nucleotides;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region;
and
b) a second oligonucleotide adapter selected from the group consisting of:
(i) an asymmetrical tail adapter comprising a first ligatable end, and a second end comprising a single-stranded 5′ overhang of at least about 8 nucleotides, wherein the 3′ end of the strand that does not comprise the 5′ overhang comprises at least one blocking group;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region;
wherein the nucleic acid sequence of the first and second double-stranded oligonucleotide adapters are not identical.

10. A method for exponential amplification of one template strand of at least one double-stranded nucleic acid molecule to produce a plurality of amplified molecules having a different sequence at each end, comprising:
a) ligating to one end of the double-stranded nucleic acid molecule a first asymmetrical adapter selected from the group consisting of:
(i) an asymmetrical tail adapter comprising a first ligatable end, and a second end comprising a single-stranded 3′ overhang of at least about 8 nucleotides;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region;
b) ligating to the other end of the double-stranded nucleic acid molecule a second asymmetrical adapter selected from the group consisting of:
(i) an asymmetrical tail adapter comprising a first ligatable end, and a second end comprising a single-stranded 5′ overhang of at least about 8 nucleotides, wherein the 3′ end of the strand that does not comprise the 5′ overhang comprises at least one blocking group;
(ii) an asymmetrical Y adapter comprising a first ligatable end, and a second unpaired end comprising two non-complementary strands, wherein the length of the non-complementary strands are at least about 8 nucleotides; and
(iii) an asymmetrical bubble adapter comprising an unpaired region of at least about 8 nucleotides flanked on each side by a paired region;
wherein the nucleic acid sequence of the first and second asymmetrical adapters are not identical,
thereby producing an end-linked double-stranded nucleic acid molecule having a first asymmetrical adapter at one end and a second asymmetrical adapter at the other end of the double-stranded nucleic acid molecule;
c) amplifying the template strand in an amplification reaction comprising a first primer and a second primer, wherein the template strand is one strand of the end-linked nucleic acid molecule, the amplification reaction comprises:
(i) contacting the template strand with a first primer, which is complementary to a first primer binding site in the first asymmetrical adapter in the template strand, under conditions in which the first primer synthesizes a first nucleic acid strand in the amplification reaction, wherein the first nucleic acid strand is complementary to the template strand, and wherein the 3′ end of the first nucleic acid strand comprises a second primer binding site that is complementary to a sequence in the second asymmetrical adapter in the template strand; and
(ii) contacting the first nucleic acid strand with a second primer which is complementary to the second primer binding site in the first nucleic acid strand under conditions in which the second primer synthesizes a complementary strand of the first nucleic acid strand,
thereby producing a plurality of exponentially amplified molecules having a different sequence at each end.

11. A method for producing and amplifying a paired tag from a first nucleic acid sequence fragment, without cloning, comprising:
a) joining the 5′ and 3′ ends of a first nucleic acid sequence fragment via a first linker such that the first linker is located between the 5′ end and the 3′ end of the first nucleic acid sequence fragment thereby producing a circular nucleic acid molecule;
b) cleaving the circular nucleic acid molecule, thereby producing a second nucleic acid sequence fragment, wherein a 5′ end tag of the first nucleic acid sequence fragment is joined to a 3′ end tag of the first nucleic acid sequence fragment via the first linker;
c) ligating a pair of asymmetrical adapters to the ends of the second nucleic acid sequence fragment, wherein the pair of asymmetrical adapters comprise:
(i) a first asymmetrical oligonucleotide adapter selected from the group consisting of:
(A) an asymmetrical tail adapter comprising a first ligatable end, and a second end comprising a single-stranded 3′ overhang of at least about 8 nucleotides;
(B) an asymmetrical Y adapter comprising a first ligatable end, and a second unpaired end comprising two non-complementary strands, wherein the length of the non-complementary strands are at least about 8 nucleotides; and
(C) an asymmetrical bubble adapter comprising an unpaired region of at least about 8 nucleotides flanked on each side by a paired region;
and
(ii) a second asymmetrical oligonucleotide adapter selected from the group consisting of:
(A) an asymmetrical tail adapter comprising a first ligatable end, and a second end comprising a single-stranded 5′ overhang of at least about 8 nucleotides, wherein the 3′ end of the strand that does not comprise the 5′ overhang comprises at least one blocking group;
(B) an asymmetrical Y adapter comprising a first ligatable end, and a second unpaired end comprising two non-complementary strands, wherein the length of the non-complementary strands are at least about 8 nucleotides; and
(C) an asymmetrical bubble adapter comprising an unpaired region of at least about 8 nucleotides flanked on each side by a paired region;
wherein the nucleic acid sequence of the first and second double-stranded oligonucleotide adapters are not identical,
thereby producing an end-linked double-stranded nucleic acid molecule having a first asymmetrical adapter at one end and a second asymmetrical adapter at the other end of the double-stranded nucleic acid molecule; and
d) amplifying the template strand in an amplification reaction comprising a first primer and a second primer, wherein the template strand is one strand of the end-linked nucleic acid molecule, the amplification reaction comprises:
(i) contacting the template strand with a first primer, which is complementary to a first primer binding site in the first asymmetrical adapter in the template strand, under conditions in which the first primer synthesizes a first nucleic acid strand in the amplification reaction, wherein the first nucleic acid strand is complementary to the template strand, and wherein the 3′ end of the first nucleic acid strand comprises a second primer binding site that is complementary to a sequence in the second asymmetrical adapter in the template strand; and
(ii) contacting the first nucleic acid strand with a second primer which is complementary to the second primer binding site in the first nucleic acid strand under conditions in which the second primer synthesizes a complementary strand of the first nucleic acid strand,
thereby producing and amplifying a paired tag from a first nucleic acid sequence fragment without cloning.

12. A method for characterizing a nucleic acid sequence, without cloning, comprising:
a) fragmenting a nucleic acid sequence thereby producing a plurality of first nucleic acid sequence fragments each having a 5′ end and a 3′ end;
b) joining the 5′ and 3′ ends of each first nucleic acid sequence fragment to a first linker such that the first linker is located between the 5′ end and the 3′ end of each first nucleic acid sequence fragment in a circular nucleic acid molecule;
c) cleaving the circular nucleic acid molecules, thereby producing a plurality of second nucleic acid sequence fragments wherein a subset of the fragments comprise a paired tag derived from each first nucleic acid sequence fragment joined via the first linker;
d) ligating a pair of asymmetrical second adapters to the ends of the second nucleic acid sequence fragment, wherein the pair of asymmetrical adapters comprise:
(i) a first asymmetrical oligonucleotide adapter selected from the group consisting of:
(A) an asymmetrical tail adapter comprising a first ligatable end, and a second end comprising a single-stranded 3′ overhang of at least about 8 nucleotides;
(B) an asymmetrical Y adapter comprising a first ligatable end, and a second unpaired end comprising two non-complementary strands, wherein the length of the non-complementary strands are at least about 8 nucleotides; and
(C) an asymmetrical bubble adapter comprising an unpaired region of at least about 8 nucleotides flanked on each side by a paired region;
and
(ii) a second asymmetrical oligonucleotide adapter selected from the group consisting of:
(A) an asymmetrical tail adapter comprising a first ligatable end, and a second end comprising a single-stranded 0.5° overhang of at least about 8 nucleotides, wherein the 3′ end of the strand that does not comprise the 5′ overhang comprises at least one blocking group;
(B) an asymmetrical Y adapter comprising a first ligatable end, and a second unpaired end comprising two non-complementary strands, wherein the length of the non-complementary strands are at least about 8 nucleotides; and
(C) an asymmetrical bubble adapter comprising an unpaired region of at least about 8 nucleotides flanked on each side by a paired region;
wherein the nucleic acid sequence of the first and second asymmetrical oligonucleotide adapters are not identical,
thereby producing an end-linked double-stranded nucleic acid molecule having a first asymmetrical adapter at one end and a second asymmetrical adapter at the other end of the double-stranded nucleic acid molecule; and
e) amplifying the template strand in an amplification reaction comprising a first primer and a second primer, wherein the template strand is one strand of the end-linked nucleic acid molecule, the amplification reaction comprises:
(i) contacting the template strand with a first primer, which is complementary to a first primer binding site in the first asymmetrical adapter in the template strand, under conditions in which the first primer synthesizes a first nucleic acid strand in the amplification reaction, wherein the first nucleic acid strand is complementary to the template strand, and wherein the 3′ end of the first nucleic acid strand comprises a second primer binding site that is complementary to a sequence in the second asymmetrical adapter in the template strand; and
(ii) contacting the first nucleic acid strand with a second primer which is complementary to the second primer binding site in the first nucleic acid strand under conditions in which the second primer synthesizes a complementary strand of the first nucleic acid strand,
thereby producing a plurality of amplified second nucleic acid fragments; and
f) characterizing the 5′ and 3′ end tags of the plurality of amplified second nucleic acid fragments.

13. A method for producing a paired end library from a nucleic acid sequence comprising:
a) fragmenting a nucleic acid sequence to produce a plurality of nucleic acid sequence fragments of an appropriate size for packaging into a lambda bacteriophage head;
b) ligating COS-linkers comprising a functional lambda bacteriophage packaging (COS) site to the plurality of nucleic acid sequence fragments under conditions in which a concatemer of nucleic acid sequence fragments and intervening COS linkers is produced;
c) packaging individual COS-linked nucleic acid sequence fragments from the concatemer into bacteriophage particles, thereby producing a plurality of packaged, circularized COS-linked nucleic acid sequences, wherein the ends of each nucleic acid sequence fragment are linked by a nicked COS site;
d) liberating the circularized COS-linked nucleic acid sequences from the bacteriophage particles under conditions that the nicked COS site remain hybridized;
e) sealing the nicked COS site in each circularized COS-linked nucleic acid sequence to produce a plurality of closed circular COS-linked nucleic acid sequences;
f) fragmenting said plurality of closed circular COS-linked nucleic acid sequences, thereby producing a paired end library from a nucleic acid sequence comprising COS-linked nucleic acid sequence fragments.

14. The method of claim 13, wherein the size of the nucleic acid fragments produced in step a) is at least about 48 kb +/−about 4 kb.

15. The method of claim 13, wherein the COS-linkers further comprise an affinity tag.

16. The method of claim 15, wherein the COS-linked nucleic acid sequence fragments are isolated by capturing the affinity tag.

17. The method of claim 15, wherein the affinity tag is selected from the group consisting of biotin, digoxigenin, a hapten, a ligand, a peptide and a nucleic acid.

18. The method of claim 13, wherein the COS-linker further comprises a selectable marker.

19. The method of claim 13, wherein said plurality of closed circular COS-linked nucleic acid sequences are fragmented in step f) by shearing.

20. The method of claim 19, wherein the plurality of closed circular COS-linked nucleic acid sequences fragmented by shearing are subsequently blunt-ended.

21. The method of claim 13, wherein said COS linker further comprises a restriction endonuclease recognition site for a restriction endonuclease that cleaves a nucleic acid sequence distally to the restriction endonuclease recognition site.

22. The method of claim 21, wherein the restriction endonuclease is a TypeIIS or Type III restriction endonuclease.

23. The method of claim 22, wherein the plurality of closed circular COS-linked nucleic acid sequences are fragmented by cleavage with a TypeIIS or Type III restriction endonuclease.

24. The method of claim 16, further comprising amplification of the isolated COS-linked nucleic acid sequence fragments, thereby producing a library of amplified COS-linked nucleic acid sequence fragments.

25. The method of claim 24, wherein the amplification comprises:
a) ligating a pair of asymmetrical adapters to the ends of each COS-linked nucleic acid sequence fragment, wherein the pair of asymmetrical adapters comprise:
(i) a first asymmetrical oligonucleotide adapter selected from the group consisting of:
(A) an asymmetrical tail adapter comprising a first ligatable end, and a second end comprising a single-stranded 3′ overhang of at least about 8 nucleotides;
(B) an asymmetrical Y adapter comprising a first ligatable end, and a second unpaired end comprising two non-complementary strands, wherein the length of the non-complementary strands are at least about 8 nucleotides; and
(C) an asymmetrical bubble adapter comprising an unpaired region of at least about 8 nucleotides flanked on each side by a paired region;
and
(ii) a second asymmetrical oligonucleotide adapter selected from the group consisting of:
(A) an asymmetrical tail adapter comprising a first ligatable end, and a second end comprising a single-stranded 5′ overhang of at least about 8 nucleotides, wherein the 3′ end of the strand that does not comprise the 5′ overhang comprises at least one blocking group;
(B) an asymmetrical Y adapter comprising a first ligatable end, and a second unpaired end comprising two non-complementary strands, wherein the length of the non-complementary strands are at least about 8 nucleotides; and
(C) an asymmetrical bubble adapter comprising an unpaired region of at least about 8 nucleotides flanked on each side by a paired region;
wherein the nucleic acid sequence of the first and second asymmetrical oligonucleotide adapters are not identical,
thereby producing an end-linked double-stranded nucleic acid molecule having a first asymmetrical adapter at one end and a second asymmetrical adapter at the other end of the double-stranded nucleic acid molecule; and
e) amplifying the template strand in an amplification reaction comprising a first primer and a second primer, wherein the template strand is one strand of the end-linked nucleic acid molecule, the amplification reaction comprises:
(i) contacting the template strand with a first primer, which is complementary to a first primer binding site in the first asymmetrical adapter in the template strand, under conditions in which the first primer synthesizes a first nucleic acid strand in the amplification reaction, wherein the first nucleic acid strand is complementary to the template strand, and wherein the 3′ end of the first nucleic acid strand comprises a second primer binding site that is complementary to a sequence in the second asymmetrical adapter in the template strand; and
(ii) contacting the first nucleic acid strand with a second primer which is complementary to the second primer binding site in the first nucleic acid strand under conditions in which the second primer synthesizes a complementary strand of the first nucleic acid strand,
thereby producing a plurality of amplified COS-linked nucleic acid fragments.

26. The method of claim 25, further comprising sequencing the plurality of amplified COS-linked nucleic acid fragments.

27. A method for producing a paired end library from a nucleic acid sequence comprising:
a) fragmenting a nucleic acid sequence to produce a plurality of nucleic acid sequence fragments of an appropriate size for packaging into a lambdoid bacteriophage head;
b) ligating COS-linkers to the plurality of nucleic acid sequence fragments under conditions in which a concatemer of nucleic acid sequence fragments and COS linkers is produced, wherein said COS-linkers comprise a functional COS site and two loxP sites flanking the functional COS site;
c) packaging individual COS-linked nucleic acid sequence fragments from the concatemer into bacteriophage particles, thereby producing a plurality of packaged, circularized COS-linked nucleic acid sequences, wherein the ends of each nucleic acid sequence fragment are linked by a nicked COS site;
d) liberating the circularized COS-linked nucleic acid sequences from the bacteriophage particles under conditions that the nicked COS site remain hybridized;
e) sealing the nicked COS site in each circularized COS-linked nucleic acid sequence to produce a plurality of closed circular COS-linked nucleic acid sequences;
f) maintaining the plurality of closed circular COS-linked nucleic acid sequences under conditions suitable for intramolecular recombination between the two loxP sites in each closed circular COS-linked nucleic acid sequence, thereby removing the functional COS site from the plurality of closed circular COS-linked nucleic acid sequence fragments, thereby producing a plurality of closed circular lox-linked nucleic acid sequences; and
g) fragmenting said plurality of closed circular lox-linked nucleic acid sequences, thereby producing a paired end library from a nucleic acid sequence comprising lox-linked nucleic acid sequence fragments.

28. The method of claim 27, wherein the size of the nucleic acid fragments produced in step a) is at least about 48 kb +/−about 4 kb.

29. The method of claim 27, wherein the COS-linkers further comprise an affinity tag.

30. The method of claim 29, wherein the lox-linked nucleic acid sequence fragments are isolated by capturing the affinity tag.

31. The method of claim 29, wherein the affinity tag is selected from the group consisting of biotin, digoxigenin, a hapten, a ligand, a peptide and a nucleic acid.

32. The method of claim 27, wherein the COS-linker further comprises a selectable marker.

33. The method of claim 27, wherein said plurality of closed circular lox-linked nucleic acid sequences are fragmented in step g) by shearing.

34. The method of claim 33, wherein the plurality of closed circular lox-linked nucleic acid sequences fragmented by shearing are subsequently blunt-ended.

35. The method of claim 27, wherein said COS linker further comprises a restriction endonuclease recognition site for a restriction endonuclease that cleaves a nucleic acid sequence distally to the restriction endonuclease recognition site.

36. The method of claim 35, wherein the restriction endonuclease is a TypeIIS or Type III restriction endonuclease.

37. The method of claim 36, wherein the plurality of closed circular lox-linked nucleic acid sequences are fragmented by cleavage with a TypeIIS or Type III restriction endonuclease.

38. The method of claim 27, wherein the two loxP sites are mutated, whereby recombination between the two loxP sites is unidirectional.

39. The method of claim 38, wherein the two loxP sites are a lox71 site and a lox66 site.

40. The method of claim 27, further comprising amplification of the isolated lox-linked nucleic acid sequence fragments, thereby producing a library of amplified lox-linked nucleic acid sequence fragments.

41. The method of claim 40, wherein the amplification comprises:
a) ligating a pair of asymmetrical adapters to the ends of each lox-linked nucleic acid sequence fragment, wherein the pair of asymmetrical adapters comprise:
(i) a first asymmetrical oligonucleotide adapter selected from the group consisting of:
(A) an asymmetrical tail adapter comprising a first ligatable end, and a second end comprising a single-stranded 3′ overhang of at least about 8 nucleotides;
(B) an asymmetrical Y adapter comprising a first ligatable end, and a second unpaired end comprising two non-complementary strands, wherein the length of the non-complementary strands are at least about 8 nucleotides; and
(C) an asymmetrical bubble adapter comprising an unpaired region of at least about 8 nucleotides flanked on each side by a paired region;
and
(ii) a second asymmetrical oligonucleotide adapter selected from the group consisting of:
(A) an asymmetrical tail adapter comprising a first ligatable end, and a second end comprising a single-stranded 5′ overhang of at least about 8 nucleotides, wherein the 3′ end of the strand that does not comprise the 5′ overhang comprises at least one blocking group;
(B) an asymmetrical Y adapter comprising a first ligatable end, and a second unpaired end comprising two non-complementary strands, wherein the length of the non-complementary strands are at least about 8 nucleotides; and
(C) an asymmetrical bubble adapter comprising an unpaired region of at least about 8 nucleotides flanked on each side by a paired region;
wherein the nucleic acid sequence of the first and second asymmetrical oligonucleotide adapters are not identical,
thereby producing an end-linked double-stranded nucleic acid molecule having a first asymmetrical adapter at one end and a second asymmetrical adapter at the other end of the double-stranded nucleic acid molecule; and
e) amplifying the template strand in an amplification reaction comprising a first primer and a second primer, wherein the template strand is one strand of the end-linked nucleic acid molecule, the amplification reaction comprises:
(i) contacting the template strand with a first primer, which is complementary to a first primer binding site in the first asymmetrical adapter in the template strand, under conditions in which the first primer synthesizes a first nucleic acid strand in the amplification reaction, wherein the first nucleic acid strand is complementary to the template strand, and wherein the 3′ end of the first nucleic acid strand comprises a second primer binding site that is complementary to a sequence in the second asymmetrical adapter in the template strand; and
(ii) contacting the first nucleic acid strand with a second primer which is complementary to the second primer binding site in the first nucleic acid strand under conditions in which the second primer synthesizes a complementary strand of the first nucleic acid strand,
thereby producing a plurality of amplified lox-linked nucleic acid fragments.

42. The method of claim 41, further comprising sequencing the plurality of amplified lox-linked nucleic acid fragments.

43. A cleavable adapter comprising an affinity tag and a cleavable linkage, wherein cleaving the cleavable linkage produces two complementary ends.

44. The cleavable adapter of claim 43, wherein the affinity tag is selected from the group consisting of biotin, digoxigenin, a hapten, a ligand, a peptide and a nucleic acid.

45. The cleavable adapter of claim 43, wherein the adapter further comprises a restriction endonuclease recognition site specific for a restriction endonuclease that cleaves a nucleic acid sequence distally to the restriction endonuclease recognition site.

46. The cleavable adapter of claim 43, wherein the cleavable linkage is a 3′ phosphorothiolate linkage.

47. The cleavable adapter of claim 43, wherein the cleavable linkage is a deoxyuridine nucleotide.

48. A method for producing a paired tag library from a nucleic acid sequence comprising:
a) fragmenting a nucleic acid sequence thereby producing a plurality of large nucleic acid sequence fragments of a specific size range;
b) introducing onto each end of each nucleic acid sequence fragment a cleavable adapter, wherein the cleavable adapter comprises an affinity tag and a cleavable linkage;
c) cleaving the cleavable adapter, thereby producing a plurality of nucleic acid sequence fragments having compatible ends;
d) maintaining the nucleic acid sequence fragments having compatible ends under conditions in which the compatible ends intramolecularly ligate, thereby producing a plurality of circularized nucleic acid sequences;
e) fragmenting the plurality of circularized nucleic acid sequences, thereby producing a plurality of paired tags comprising a linked 5′ end tag and a 3′ end tag of each nucleic acid sequence fragment,
thereby producing a paired tag library from a plurality of large nucleic acid sequence fragments.

49. The method of claim 48, wherein the specific size range of the large nucleic acid fragments in step a is from about 2 to about 200 kilobase pairs.

50. The method of claim 48, wherein the large nucleic acid sequence fragments are produced by shearing.

51. The method of claim 48, wherein the plurality of circularized nucleic acid sequences in step e) are sheared to produce the plurality of paired tags comprising a linked 5′ end tag and a 3′ end tag of each nucleic acid sequence fragment.

52. The method of claim 51, wherein the plurality of paired tags comprising a linked 5′ end tag and a 3′ end tag of each nucleic acid sequence fragment are blunt-ended.

53. The method of claim 48, wherein the cleavable adapter further comprises a restriction endonuclease recognition site specific for a restriction endonuclease that cleaves a nucleic acid sequence distally to the restriction endonuclease recognition site.

54. The method of claim 53, wherein the plurality of circularized nucleic acid sequences in step e) are cleaved by a restriction endonuclease that cleaves the nucleic acid sequence fragment distally to the restriction endonuclease recognition site.

55. The method of claim 48, wherein the affinity tag is selected from the group consisting of biotin, digoxigenin, a hapten, a ligand, a peptide and a nucleic acid.

56. The method of claim 48, wherein the method further comprises isolating the plurality of paired tags comprising the linked 5′ end tag and a 3′ end tag of each nucleic acid sequence fragment by capturing the affinity tags, thereby producing an isolated paired tag library.

57. The method of claim 56, wherein the method further comprises amplification of said isolated paired tag library to produce a library of amplified paired tags.

58. The method of claim 57, wherein said amplification comprises:
a) ligating a pair of asymmetrical adapters to the ends of each paired tag, wherein the pair of asymmetrical adapters comprise:
(i) a first asymmetrical oligonucleotide adapter selected from the group consisting of:
(A) an asymmetrical tail adapter comprising a first ligatable end, and a second end comprising a single-stranded 3′ overhang of at least about 8 nucleotides;
(B) an asymmetrical Y adapter comprising a first ligatable end, and a second unpaired end comprising two non-complementary strands, wherein the length of the non-complementary strands are at least about 8 nucleotides; and
(C) an asymmetrical bubble adapter comprising an unpaired region of at least about 8 nucleotides flanked on each side by a paired region;
and
(ii) a second asymmetrical oligonucleotide adapter selected from the group consisting of:
(A) an asymmetrical tail adapter comprising a first ligatable end, and a second end comprising a single-stranded 5′ overhang of at least about 8 nucleotides, wherein the 3′ end of the strand that does not comprise the 5′ overhang comprises at least one blocking group;
(B) an asymmetrical Y adapter comprising a first ligatable end, and a second unpaired end comprising two non-complementary strands, wherein the length of the non-complementary strands are at least about 8 nucleotides; and
(C) an asymmetrical bubble adapter comprising an unpaired region of at least about 8 nucleotides flanked on each side by a paired region;
wherein the nucleic acid sequence of the first and second asymmetrical oligonucleotide adapters are not identical,
thereby producing a library of end-linked paired tags having a first asymmetrical adapter at one end and a second asymmetrical adapter at the other end of the paired tags; and
b) amplifying the template strand in an amplification reaction comprising a first primer and a second primer, wherein the template strand is one strand of the end-linked nucleic acid molecule, the amplification reaction comprises:
(i) contacting the template strand with a first primer, which is complementary to a first primer binding site in the first asymmetrical adapter in the template strand, under conditions in which the first primer synthesizes a first nucleic acid strand in the amplification reaction, wherein the first nucleic acid strand is complementary to the template strand, and wherein the 3′ end of the first nucleic acid strand comprises a second primer binding site that is complementary to a sequence in the second asymmetrical adapter in the template strand; and
(ii) contacting the first nucleic acid strand with a second primer which is complementary to the second primer binding site in the first nucleic acid strand under conditions in which the second primer synthesizes a complementary strand of the first nucleic acid strand,
thereby producing an amplified library of paired tags.

59. The method of claim 58, further comprising sequencing the amplified library of paired tags.

60. The method of claim 48, wherein the nucleic acid sequence is a genome.

61. The method of claim 48, wherein the cleavable linkage in the cleavable adapter is a 3′ phosphorothiolate linkage.

62. The method of claim 48, wherein the cleavable linkage in the cleavable adapter is a deoxyuridine nucleotide.

63. The method of claim 61, wherein the 3′ phosphorothiolate linkage is cleaved by Ag+, Hg2+ or Cu2+, at a pH of at least about 5 to at least about 9, and at a temperature of at least about 22° C. to at least about 37° C.

64. The method of claim 62, wherein the deoxyuridine is cleaved by uracil DNA glycosylase (UDG) and an AP-lyase.


SEQUENCE LISTING (26 sequences)

SEQ ID NO: 1 - DNA, 209 bp, Artificial Sequence
  Minimal Lambda phage COS site
tcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgaggctttttggcctctgtcgtttcctttctctgt

SEQ ID NO: 2 - DNA, 27 bp, Artificial Sequence
  CosN recognition site and flanking sequence
acaggttacggggcggcgacctcgcgg

SEQ ID NO: 3 - DNA, 31 bp, Artificial Sequence
  Primer
ctgctgtcactttacgggtcctttccggtga

SEQ ID NO: 4 - DNA, 43 bp, Artificial Sequence
  Primer
ctgctgacagagaaaggaaacgacagaggccaaaaagctcgct

SEQ ID NO: 5 - DNA, 59 bp, Artificial Sequence
  Primer
ctgctgtcgtataatgtatgctatacgaacggtatcactttacgggtcctttccggtga

SEQ ID NO: 6 - DNA, 71 bp, Artificial Sequence
  Primer
ctgctgattgaagcatatcgtatgtaatatgcttacagagaaaggaaacgacagaggccaaaaagctcgct

SEQ ID NO: 7 - DNA, 209 bp, Artificial Sequence; identical to SEQ ID NO: 1
  COS linker

SEQ ID NO: 8 - DNA, 13 bp, Artificial Sequence
  Restriction endonuclease recognition site
  N_region (4)...(9): n = any nucleotide
aacnnnnnngtgc

SEQ ID NO: 9 - DNA, 14 bp, Artificial Sequence
  Restriction endonuclease recognition site, upperstrand
  N_region (6)...(14): n = any nucleotide
ggatgnnnnnnnnn

SEQ ID NO: 10 - DNA, 34 bp, Artificial Sequence
  Restriction endonuclease recognition site, upperstrand
  N_region 1-10, 14-19, 23-34: n = any nucleotide
nnnnnnnnnncgannnnnntcgnnnnnnnnnnnn

SEQ ID NO: 11 - DNA, 34 bp, Artificial Sequence
  Restriction endonuclease recognition site, lowerstrand
  N_region 1-10, 14-19, 23-34: n = any nucleotide
nnnnnnnnnngcannnnnntcgnnnnnnnnnnnn

SEQ ID NO: 12 - DNA, 32 bp, Artificial Sequence
  Restriction endonuclease recognition site
  N_region (7)...(32): n = any nucleotide
cagcagnnnnnnnnnnnnnnnnnnnnnnnnnn

SEQ ID NO: 13 - DNA, 22 bp, Artificial Sequence
  Restriction endonuclease recognition site, upperstrand
  N_region (7)...(22): n = any nucleotide
ctgaagnnnnnnnnnnnnnnnn

SEQ ID NO: 14 - DNA, 20 bp, Artificial Sequence
  Restriction endonuclease recognition site, lowerstrand
  N_region (1)...(14): n = any nucleotide
nnnnnnnnnnnnnncttcag

SEQ ID NO: 15 - DNA, 12 bp, Artificial Sequence
  Asymmetric tail adapter, AsymA1
ctctcgtcttgc

SEQ ID NO: 16 - DNA, 78 bp, Artificial Sequence
  Asymmetric tail adapter, AsymA2
gcaagacgagaggtcccacacgtaacaccaaacctatccacacttttacaaaccactaggacagtcgctaccttagtg

SEQ ID NO: 17 - DNA, 50 bp, Artificial Sequence
  Asymmetric tail adapter, AsymA3
gcaagacgagaggtcccacacgtaacactaggacagtcgctaccttagtg

SEQ ID NO: 18 - DNA, 28 bp, Artificial Sequence
  Asymmetric tail adapter, AsymA4
gtgttacgtgtgggacctctcgtcttgc

SEQ ID NO: 19 - DNA, 14 bp, Artificial Sequence
  Asymmetric tail adapter, AsymB1
  modified_base (12)...(14)
catcctactctccc

SEQ ID NO: 20 - DNA, 50 bp, Artificial Sequence
  Asymmetric tail adapter, AsymB2
ccttaggaccgttatagttaggtgcagaagcgaacacagagagtaggatg

SEQ ID NO: 21 - DNA, 36 bp, Artificial Sequence
  Asymmetrical tail adapter, AsymB3
ccttaggaccgttatagttaggtggagagtaggatg

SEQ ID NO: 22 - DNA, 26 bp, Artificial Sequence
  Asymmetrical tail adapter, AsymB4
  modified_base (24)...(26)
catcctactctctgtgttcgcttccc

SEQ ID NO: 23 - DNA, 24 bp, Artificial Sequence
  Adapter, upper strand
  misc_feature (6)...(7)
ggagctgtacaacgacacctagac

SEQ ID NO: 24 - DNA, 22 bp, Artificial Sequence
  Adapter, lower strand
  misc_feature (18)...(19)
gtctaggtgtcgttgtacagct

SEQ ID NO: 25 - DNA, 18 bp, Artificial Sequence
  Restriction endonuclease recognition site, lowerstrand
  N_region (1)...(13): n = any nucleotide
nnnnnnnnnnnnncatcc

SEQ ID NO: 26 - DNA, 209 bp, Artificial Sequence; identical to SEQ ID NO: 1
  COS linker